(19) United States
(12) Patent Application Publication
(10) Pub. No.: US 2023/0398294 A1
(43) Pub. Date: Dec. 14, 2023

(54) Electronic system for a drug delivery device and drug delivery device

(71) Applicant: Sanofi, PARIS (FR)

(72) Inventors: Stefan Alt, Frankfurt am Main (DE); Oliver Charles Gazeley, Basel (CH); Adam Moyo Harvey-Cook, Billericay, Essex (GB); Stephen Ingram, Warwick, Warwickshire (GB); Craig Ashley Mason, Helsby, Cheshire (GB); Aidan Michael O`Hare, Warwick, Warwickshire (GB); David Aubrey Plumptre, Worcestershire (GB)

(21) Appl. No.: 18/034,523

(22) PCT Filed: Oct. 27, 2021
(86) PCT No.: PCT/EP2021/079747

§ 371 Date: Apr. 28, 2023

(30) Foreign Application Priority Data
Oct. 30, 2020 (EP) 20315440.6

(51) Int. Cl. A61M 5/172

(52) U.S. Cl. CPC A61M 5/1723; A61M 2205/50

(57) ABSTRACT

An electronic system for a drug delivery device includes a user interface member configured for manipulation by a user to perform a dose operation. An electronic control unit is configured to control operation of the electronic system. The electronic system has a first state and a second state that has an increased electrical power consumption as compared to the first state. An electrical signaling unit is configured to provide an electrical signal indicative that the user interface member is being manipulated and to switch the electronic system from the first state into the second state in response to the electrical signal. The user interface member comprises a first portion that is movable relative to a second portion from a rest position to a signaling position. The signaling unit is configured to provide the electrical signal in response to the movement of the first portion relative to the second portion.

### CROSS REFERENCE TO RELATED APPLICATIONS

The present application is the national stage entry of International Patent Application No. PCT/EP2021/079747, filed Oct. 27, 2021, and claims priority to Application No. EP 20315440.6, filed on Oct. 30, 2020, the disclosures of which are incorporated herein by reference.

### TECHNICAL FIELD

The present disclosure relates to an electronic system for a drug delivery device. The present disclosure further relates to a drug delivery device, which preferably comprises the electronic system.

### BACKGROUND

Drug delivery devices using electronics are becoming increasingly popular in the pharmaceutical industry as well as for users or patients. However, especially if the device is designed to be self-contained, that is to say without a connector for a connection to an external electrical power source which is necessary to provide electrical power for the operation of the device, the management of the resources of a power supply integrated into the device is particularly important.

### SUMMARY

It is an object of the present disclosure to provide a novel, expediently an improved electronic system.

This object is achieved by the subject-matter of the independent claim. Advantageous embodiments and refinements are subject to the dependent claims. We note that the present disclosure is not restricted to what is currently claimed and may contain subject matter only in the description which could be made subject to claims.

One aspect of the present disclosure relates to an electronic system for a drug delivery device. Another aspect of the present disclosure relates to a drug delivery device, especially one comprising the electronic system. Accordingly, the features, which are disclosed in relation to the drug delivery device or units thereof or therefore, do also apply for the electronic system and vice versa.

In one embodiment, the electronic system comprises at least one user interface member. The user interface member may be arranged or configured to be manipulated, e.g. touched and/or moved, by a user, e.g. the user of the drug delivery device. The user interface member may be provided for performing a dose operation, e.g. a dose setting operation to set a dose of drug to be delivered by the drug delivery device and/or for performing a dose delivery operation for delivering a set dose, preferably the dose one which has been previously set during the dose setting operation. The dose operation may be an operation which is related to the dose which is to be delivered by the drug delivery device, either to the delivery operation or to the dose setting operation. The dose operation, in addition to the dose setting operation, may comprise a dose adjusting operation, where a previously set dose is either increased or decreased by manipulating the user interface member. The dose operation may be performed by the user of the drug delivery device, e.g. a patient. The dose operation may require continuous contact of the user to the user interface member throughout the entire operation. The dose operation may involve movement of the user interface member relative to a housing of the drug delivery device, e.g. a housing of a drug delivery device unit to which the electronic system should be connected or a housing of the drug delivery device. Alternatively or additionally to the dose operation the user interface member may be manipulated for an activation operation. The activation operation may be provided alternatively or additionally to all dose operations, that is to say alternatively or additionally to the dose setting operation and the dose delivery operation and, if applicable, to a dose adjusting or cancelling operation.

In one embodiment, the user interface member has an exterior operation surface arranged and configured to be touched by the user. The exterior operation surface may be arranged and configured to be touched during operation of the system or the device, such as the dose operation, e.g. for initiating and/or for performing the operation. Thus, the exterior operation surface may be or may comprise a setting surface for the setting operation and/or a delivery surface for the delivery operation. The setting surface and the delivery surface may face in different directions. The setting surface may face in the radial or lateral direction. The delivery surface may face in an axial, e.g. in a proximal, direction. The setting surface may extend circumferentially around an axis of the user interface member. The delivery surface may be arranged obliquely or perpendicularly relative to the axis.

In one embodiment, the electronic system comprises an electronic control unit. The electronic control unit may be configured to control an operation of the electronic system. The electronic control unit may be or may comprise an electronic processor, such as a microcontroller or an ASIC, for example. The electronic system may have a first state, e.g. when the system is dormant or idle, and a second state, e.g. when the system is operational. The electronic system may have an increased electrical power consumption in the second state as compared to the first state. In the first state, one or more electrical or electronic units of the electronic system may be in a sleep mode or be powered off such that they have no significant power consumption or no power consumption. For example, in the second state, a motion sensing unit may be active, i.e. it can be operated, where the motion sensing unit is not active, i.e. it cannot be operated, in the first state. The motion sensing unit will be described in more detail below. Alternatively or additionally, a communication unit may be inactive in the first state and active in the second state. The communication unit will be described in more detail below.

In one embodiment, the electronic system comprises an electrical signaling unit. The unit may be configured to provide at least one electrical signal when the user interface member is manipulated. The signal may be indicative that the user interface member is being manipulated. For example, the at least one electrical signal may be initiated or generated when the user commences movement of the user interface member or attempts to move the user interface member, for example relative to the housing. The electrical signal may be indicative for a dose operation, e.g. for a dose setting operation or a dose delivery operation, or the activation operation. The signal may be indicative that the dose operation is being commenced or that the activation operation has been completed. The housing may be the housing of the drug delivery device or of the drug delivery device unit to which the electronic system should be connected. Movement of at least a part or portion of the user interface member or only a part or portion of the user interface member may be required to generate the respective signal. This is explained in more detail below. The signaling unit may be configured to generate or provide a signal indicative for the dose operation and/or for the activation operation.

In one embodiment, the electronic control unit is configured to switch the electronic system from the first state into the second state, e.g. by issuing an according command or signal to other units or components of the system. The electronic control unit may be configured to switch the electronic system from the first state into the second state in response to the at least one electrical signal. The signal may be indicative for the dose operation or may require a dedicated operation different from the dose operation such as the activation operation. The signal indicative for the activation operation may be used, for example, to selectively activate the communication unit e.g. without activating the motion sensing unit.

In one embodiment, the user interface member comprises a first portion and a second portion. The first portion may exhibit or provide the exterior operation surface. The second portion may exhibit or provide an interface of the user interface member to a dose setting and/or drive mechanism of the drug delivery device or the drug delivery device unit. The interface may establish a permanent or non-releasable connection to the dose setting and/or drive mechanism, e.g. in case the system is integrated into the device, or a releasable connection to the dose setting and/or drive mechanism, e.g. in case the system is an add-on module for the mechanism. The second portion may provide a connection feature for connection of the user interface member to the drug delivery device unit, e.g. a snap feature. The system may be axially and/or rotationally locked to the dose setting and/or drive mechanism, e.g. one member thereof by the connection. The dose operation may require a movement of the second portion relative to the housing. The activation operation may require a movement of the first portion relative to the second portion, e.g. from a rest position of the first portion to a signaling position of the first portion relative to the second portion. The movement of the first portion relative to the second portion into the signaling position may be required to trigger the signaling unit to provide the electrical signal. The second portion may be stationary during the relative movement of the first portion from the rest position to the signaling position. That is to say the second portion may not move relative to a housing for example. The first portion may be movable relative to the second portion from the rest position of the first portion to a signaling position of the first portion relative to the second portion. The signaling unit may be configured to provide the electrical signal in response to the displacement or movement of the first portion relative to the second portion from the rest position into the signaling position. Thus, the relative movement may be required for providing the electrical signal. Completing the dose operation may involve or require simultaneous movement of the first and second portions, e.g. after the first portion has reached the signaling position relative to the second portion. The rest position may be a position which the first portion has relative to the second portion when no force or torque is applied to the user interface member by the user. When the first portion is in the rest position, the user interface member may be in an initial position, i.e. a position before the dose operation is or can be commenced.

Using relative movement between the first and second portions, which are both part of the user interface member, integrates the process of generating the signal which causes switching of electronic system into the second state into the manipulation of the user interface member without involving another member to activate the system, e.g. to wake-up the system such as by switching the system from a dormant state into a functional state.

In one embodiment, for the activation operation, the user manipulates the setting surface of the user interface member, preferably only the setting surface. The activation operation may involve rotational movement of the first portion relative to the second portion, e.g. counter to a dose setting direction.

In one embodiment, for the activation operation, the user manipulates the delivery surface of the user interface member, preferably only the delivery surface. The activation operation may involve axial movement of the first portion relative to the second portion, e.g. in a dose delivery direction.

In one embodiment, the electronic control unit is connected, e.g. mounted, to the second portion. The electronic control unit may be fixedly connected to the second portion of the user interface member. The electronic control unit may be mounted on a conductor carrier and/or conductively connected to conductors on the conductor carrier. The conductor carrier may be fixed to the second portion of the user interface member. Consequently, during the movement of the first portion relative to the second portion, the first portion may move relative to the electronic control unit and/or the conductor carrier. As the electronic control unit is mounted to that part of the user interface member which the first portion moves relative to when moving from the rest position to the signaling position, the signal generation can occur close to the electronic control unit. This offers the opportunity to have short conduction paths to the electronic control unit. Moreover, the connection to the second portion ensures that the electronic control unit does not have to be moved by the user when the first portion is moved relative to the second portion.

In one embodiment, the movement from the rest position to the signaling position of the first portion relative to the second portion involves rotational movement of the first portion relative to the second portion, preferably only rotational movement that is to say without an axial movement of the first portion relative to the second portion. Rotational movement may occur when the user manipulates the setting surface of the user interface member, e.g. for the dose setting operation or the activation operation. Such a manipulation usually occurs before the delivery operation is conducted, which typically involves the delivery surface being touched by the user, and, therefore, after having been switched to the second state, the system is given sufficient time such that the electronic components or units of the system may be woken or rendered operational before the delivery operation is commenced.

In one embodiment, the movement from the rest position to the signaling position of the first portion relative to the second portion involves axial movement of the first portion relative to the second portion, preferably only axial movement, that is to say without a rotational movement of the first portion relative to the second portion. In this case, the user may manipulate the delivery surface of the user interface member. The system may be switched to the second state immediately before or when the delivery operation is being commenced. Thus, electronic functionalities of the system may be provided only when they are needed, such as during the dose delivery operation.

In one embodiment, the second portion is stationary, e.g. relative to the housing, when the first portion moves from the rest position into the signaling position. Movement of the second portion into the same direction as the first portion, e.g. relative to the housing, may be blocked. The blocking may be achieved, for example, by a stop feature.

In one embodiment, for conducting the dose operation, the first and the second portion have to move, e.g. relative to the housing. The first and the second portion may have to move in unison, expediently rotationally and axially, for the dose operation. The second portion may be slaved to the first portion for the dose operation.

In one embodiment, the force which has to be overcome for actuating movement of the second portion relative to the housing, e.g. for the dose setting operation or the dose delivery operation may comprise frictional force and/or other components such as a biasing force which needs to be overcome to switch a clutch coupling in the drug delivery device or the forces involved in activating the mechanism, i.e. before the delivery operation can be commenced.

In one embodiment, the first portion and the second portion act as one integral portion during the dose operation. The user interface member may act as a rigid single member during the dose operation(s), i.e. without relative movement between the portions.

In one embodiment, the relative movement, e.g. axial and/or rotational movement, between the first portion and the second portion of the user interface member from the rest position to the signaling position is limited, e.g. unidirectionally or bidirectionally.

In one embodiment for setting a dose in a dose setting operation, the user interface member, e.g. the second portion thereof, has to be rotated relative to the housing, e.g. in a dose setting direction. The rotation may be a rotation in whole number multiples of a unit setting increment angle. The unit setting increment may be the smallest dose which can be set to be delivered by the drug delivery device. The unit setting increment angle may be the angle by which the user interface member, e.g. its second portion and/or its first portion, may have to be rotated, e.g. relative to the housing. The angle by which the first portion is rotated relative to the second portion when the first portion moves into the signaling position may be less than the angle corresponding to one unit increment, i.e. the unit setting increment angle. For example, the angle which separates the rest position and the signaling position angularly may be less than or equal to half the unit setting increment angle.

In one embodiment, if the first portion rotates relative to the second portion from the rest position to the signaling position, the angular distance between the rest position and the signaling position is less than or equal to one of the following values: 10°, 8°, 7°, 6°, 5°, 4°, 3°, 2°, 1°. The distance may be greater than 0.5°.

In one embodiment, if the first portion moves axially relative to the second portion from the rest position to the signaling position, this movement may be limited to distances below any one of the following values: 1 mm, 0.5 mm, 0.3 mm. The movement may be greater than or equal to 0.2 mm.

In one embodiment, the distance of a relative axial movement between the first portion and the second portion from the rest position to the signaling position may be less than the distance by which the user interface member has to be moved relative to the housing, in order to switch a clutch coupling in the dose setting and/or drive mechanism, e.g. to switch the mechanism from a dose setting configuration for the dose setting operation to a dose delivery configuration for the dose delivery operation. Thus, the signaling position may be reached before the configuration of the mechanism is changed.

In one embodiment, the first portion and the second portion are mechanically interconnected during the dose operation, e.g. only during the dose operation, by a mechanical interconnection. The first portion and the second portion may be directly interconnected by the mechanical interconnection during the dose operation. The mechanical interconnection may be configured to transmit force or torque from the first portion to the second portion during the dose operation. The mechanical interconnection may be a permanent connection, e.g. a connection that is not releasable without permanently destroying features involved in establishing the connection, or a releasable connection. Alternatively, the mechanical interconnection may be releasable. For example, the mechanical interconnection may be released when the user performs an activation operation with the user interface member in order to switch the electronic system from the first state into the second state. For the dose operation, e.g. the dose setting operation and the dose delivery operation, the mechanical interconnection may be established.

In one embodiment, the mechanical interconnection comprises at least one connection feature. The user interface member may comprise a plurality of connection features. The connection feature may be configured to provide the mechanical interconnection. The connection feature may be integrally formed with the first portion and/or the second portion. The mechanical interconnection may comprise only one connection feature which is integrally formed with the first portion and the second portion or a plurality of connection features, each being integrally formed with the first portion and the second portion. A connection feature integrally formed with the first portion and the second portion may be or may comprise a web which extends between the first and second portion. The (respective) connection feature may be oriented radially. The connection feature may radially interconnect the first and the second portion with one another.

In one embodiment, a transition region between the connection feature and the first portion and/or a transition region between the connection feature and the second portion may be rigid. The connection feature may be flexible. Particularly, the connection feature may provide the flexibility for permitting movement of the first portion relative to the second portion from the rest position to the signaling position. The connection feature may be elastically deformable. The connection feature may be elastically deformed when the first portion is moved from the rest position to the signaling position.

In one embodiment, the mechanical interconnection comprises at least one connection feature of the first portion and at least one connection feature of the second portion. The connection features are expediently configured to interact with each other, e.g. to engage one another, in order to establish the mechanical interconnection. The respective connection feature may be rigid. When the connection features of the first and second portion are engaged, the mechanical interconnection may be formed. When the connection features are disengaged, the mechanical interconnection may be released. The connection features may be snap features, for example.

In one embodiment, the first portion and the second portion are coupled via a force-sensitive coupling. The coupling may lock the portions against relative movement, e.g. against relative rotational movement, until a force acts on the coupling which exceeds a threshold force. Then, the coupling may be released and the first portion may move relative to the second portion. Forces for the dose operation, e.g. forces for setting or canceling doses are preferably reliably transferred via the force-sensitive coupling. The connection features of the mechanical interconnection may establish the force-sensitive coupling.

In one embodiment, the system is configured such that the first portion is connected to the second portion in a torque-proof and/or force-proof way after the relative movement has been completed in the direction away from the rest position, e.g. into the signaling position or beyond the signaling position, such as by a rotation by less than one unit increment angle. This may be achieved by an interaction feature on the first portion abutting an interaction feature on the second portion. Therefore, the mechanical interconnection may not be additionally stressed for transferring load from the first portion to the second portion after the signaling position has been reached.

In one embodiment, the dose operation is a dose setting operation. Alternatively, the dose operation may be a dose delivery operation.

In one embodiment, the dose setting operation involves a rotational movement of the first portion relative to a housing, e.g. the housing of the drug delivery device, in a dose setting direction.

In one embodiment, the movement of the first portion away from the rest position involves rotational movement in or counter to the dose setting direction.

In one embodiment, the second portion is also moved during the dose operation along with the first portion. The entire user interface member may be moved during the dose operation. During the initial phase of the movement for the dose operation, the first portion may be moved into the signaling position and, thereafter, the first and the second portions may be moved together. In the dose setting operation, the entire user interface member be moved relative to the housing from an initial position to a dose set position. In the initial position, the first portion may be in its rest position.

In one embodiment, the first portion and the second portion move together, e.g. rotate together, preferably in unison, when the movement of the first portion is continued from the signaling position in a direction away from the rest position. The direction away from the signaling position and the rest position may be the dose setting direction. The second portion may be slaved to follow the first portion once the signaling position has been reached, e.g. by the first portion abutting the second portion.

In one embodiment, the mechanical interconnection is releasable. The mechanical interconnection may be releasable for conducting the activation operation. The activation operation may involve movement of the first portion relative to the second portion, particularly from the rest position into the signaling position. The activation operation may be different from the dose operation as has been discussed above already. The movement may be rotational movement counter to the dose setting direction.

In one embodiment, the electronic system, preferably the mechanical interconnection, is configured such that the force or torque which the user has to exert on the first portion to perform the dose operation, e.g. to move the first portion and the second portion, with the user interface member is less than the force or torque which the user has to exert on the first portion in order to move the first portion from the rest position to the signaling position, e.g. to perform the activation operation. The force or torque necessary for the dose operation may be determined by the force required to move the second portion for the dose operation, e.g. relative to a housing and/or one or more members of the dose setting and/or drive mechanism. As the mechanical interconnection may have to be released in order to move the first portion into the signaling position from the rest position, the force or torque which the user has to exert for the movement into the signaling position may have to be greater than the force or torque for the dose operation, particularly the dose setting operation.

In one embodiment, the electronic system, preferably the mechanical interconnection, is configured such that the force or torque which the user has to exert on the first portion to perform the dose operation, e.g. to move the second portion along with the first portion, with the user interface member is greater than the force or torque which the user has to exert on the first portion in order to move the first portion from the rest position to the signaling position.

In one embodiment, the system is configured such that the second portion is configured to abut a stop feature when the first portion is moved from the rest position towards the signaling position. This facilitates or enables movement of the first portion relative to the second portion into the signaling position. The stop feature may be arranged to block movement of the second portion into the same direction as the first portion moves towards the signaling position. This facilitates an increase in the force or torque which the user exerts on the mechanical connection, e.g. to either flex the connection feature or release the mechanical interconnection, in order to move the first portion relative to the second portion into the signaling position. The stop feature may be provided in the drug delivery device or in the drug delivery device unit, e.g. locked with respect to the housing.

In one embodiment, for the dose setting operation, the first portion is moved away from the signaling position and away from the rest position. In this case, the activation operation is expediently an operation different from the dose setting operation.

In one embodiment, for the dose setting operation or the dose delivery operation, the first portion is moved away from the rest position and towards the signaling position. In this case, the signal generation may be integrated into the regular dose setting operation without a separate activation operation being required.

In one embodiment, the signaling unit is configured such that the electrical signal is only provided if the first portion is moved from the rest position to the signaling position when the size of the currently set dose is zero. When no dose is set, the user interface member may be in the position closest to the housing, e.g. in a dose setting configuration of the dose setting and/or drive mechanism. Alternatively, the signaling unit may provide the signal independently of whether a non-zero dose is currently set or not.

In one embodiment, the rest position is a zero dose or no dose set position of the first portion and/or the user interface member. That is to say, in the rest position, the size of the currently set dose may be zero. The rest position may be a position of the user interface member and/or the first portion which is as close to the housing of the drug delivery device or the drug delivery device unit as possible, e.g. when the mechanism is in the dose setting configuration. For example, when the first portion is in the rest position relative to a housing, the user interface member may be in the initial position before the dose setting operation is commenced.

In one embodiment, the signaling unit comprises an electrical switch mechanism. The switch mechanism may be configured to be triggered, preferably to provide the electrical signal, when the first portion is moved relative to the second portion from the rest position into the signaling position. An electrical switch of the switch mechanism may be arranged on the conductor carrier and/or fixed to the second portion. The switch may be conductively connected to the electronic control unit. The switch mechanism may be configured such that the electronic control unit can determine the rotation direction of the first portion relative to the second portion, e.g. distinguish between two opposite directions. The switch mechanism may be configured to generate a signal only for a unidirectional rotation or for a bidirectional rotation, e.g. by having multiple switches, one for each direction.

In one embodiment, the electronic system is configured such that, when the first portion is in the signaling position, the first portion is biased towards the rest position, e.g. by a biasing mechanism of the user interface member. The user who may manipulate the user interface member may react the bias such that the signaling position of the first portion is maintained by the user. However, once the user releases the user interface member, the biasing mechanism may be configured to reestablish the relative arrangement of the first of the second portion such that the first portion, again, assumes the rest position relative to the second portion. The biasing mechanism may comprise an elastically displaceable or deformable feature which is elastically displaced or deformed and, thereby, biased when the first portion is moved into the signaling position. The elastic restoring force provided by the feature moves the first portion, particularly from the signaling position, back into the rest position. The feature may be a flexible arm, for example. Alternatively or additionally, the elastically displaceable or deformable feature is a connection feature of the mechanical interconnection.

In one embodiment, the second portion is an internal portion of the user interface member. Preferably, the second portion is inaccessible from the exterior of the electronic system, e.g. when the electronic system is integrated into a drug delivery device or connected to a drug delivery device unit. In the electronic system, the second portion may be inaccessible from the lateral surface or the proximal surface of the user interface member. It may be accessible from a distal end of the user interface member, e.g. for being connected to a member of the dose setting and/or drive mechanism.

In one embodiment, the signaling position is angularly offset from the rest position in a direction which is opposite to the dose setting direction. For example, a rotation required for rotating the first portion relative to the second portion into the signaling position may be counter to the dose setting direction.

In one embodiment, a stop feature may be provided to block rotation of the first portion relative to the second portion, e.g. beyond the signaling position. Limited rotatability of the first portion relative to the second portion is expediently allowed, however.

In one embodiment, the movement of the first portion relative to the second portion into the signaling position in order to provide the electrical signal which is causal for switching the electronic system from the first state into the second state is unidirectional. Complicated multidirectional movements may be avoided in this way.

In one embodiment, the first portion and the second portion are rigid. The connection feature between the portions may be flexible.

In one embodiment, the user interface member is movable, e.g. axially, from a first position to a second position, for example relative to the housing, such as the housing of the system or the drug delivery device. The first position may be an initial position which the user interface member has relative to the housing, e.g. before a dose delivery operation is commenced and/or after the dose setting operation has been completed. The second position may be a position which the user interface member assumes when a user force is applied to the user interface member, e.g. a distally directed force, and the user interface member is moved away from the first position, e.g. for the dose delivery operation. The first position and the second position may be axially offset. The movement from the first position to the second position may involve only axial movement.

In one embodiment, the first portion and the second portion are operatively coupled to one another by a guiding interface. The guiding interface may be provided additionally to the connection feature and/or the mechanical interconnection. The guiding interface may guide the movement of the first portion relative to the second portion from the rest position into the signaling position, e.g. axially. The guiding interface may be configured to prevent a tilting movement of the first portion relative to the second portion, e.g. relative to a rotation axis of the first portion and/or the user interface member for the first operation. The rotation axis may be the axis of the user interface member and/or the rotation axis around which the user interface member is rotated for the dose setting operation.

In one embodiment, the electronic system or the drug delivery device comprises a dose setting and/or drive mechanism. The dose setting and/or drive mechanism may comprise a first member and a second member. The first member and/or the second member may be configured to move during the dose setting operation and/or the dose delivery operation relative to the housing of the electronic system or the drug delivery device. The first member may be a dose member or dial member of the dose setting and/or drive mechanism, which is moved to set a dose, e.g. a dial sleeve or a number sleeve. The second member may be a drive member, e.g. a member engaged with a piston rod of the dose setting and/or drive mechanism, or a device user interface member, such as a dose knob and/or injection button. The first member and/or the second member may be movably coupled to or retained in the housing. In the dose setting operation, the first member and/or the second member may be displaced axially relative to the housing, for example away from a proximal end of the housing. The distance by which the first member and/or the second member is displaced during the dose setting operation relative to the housing, e.g. axially, may be determined by the size of the set dose. In other words, the drug delivery device may be of the dial extension type, i.e. the device increases its length during the dose setting operation in an amount proportional to the size of the set dose.

In one embodiment, in the dose setting operation and/or in the dose delivery operation, the first member moves, e.g. rotates and/or moves axially, relative to the second member. For example, the first member may rotate relative to the second member during the dose delivery operation, e.g. only during the dose delivery operation. The first member and the second member may both move axially during the dose delivery operation. The first member may rotate relative to the second member and relative to the housing during the dose setting operation and/or the dose delivery operation. The second member may be rotationally locked or guided with respect to the housing during the dose delivery operation, e.g. by a delivery clutch. The first member and the second member may be rotationally locked relative to one another during the dose setting operation. Accordingly, the first member and the second member may rotate relative to the housing in the dose setting operation. During the dose setting operation the first member and the second member may be coupled to one another, e.g. via a coupling interface, e.g. a setting clutch. The coupling interface may rotationally lock the first member and the second member to one another during the dose setting operation. When the coupling interface is engaged, the first member and the second member may be rotationally locked with one another, such as by direct engagement of coupling interface features. The first member and the second member may comprise mating coupling interface features. The coupling interface may be released during the dose delivery operation, e.g. by axially displacing the second member relative to the first member. Hence, the second member may be rotationally locked relative to the housing during dose delivery, whereas the first member may rotate relative to the housing during dose delivery. The coupling interface may be released when switching the dose setting and/or drive mechanism from a dose setting configuration into a dose delivery configuration. This may be achieved when the user interface member is moved from the first position to the second position. In the first position, the mechanism may be in the dose setting configuration. In the second position, the mechanism may be in the dose delivery configuration.

In one embodiment, the first member and the second member rotate relative to one another during only one of the dose setting operation and the dose delivery operation. One of the first member and the second member, e.g. the first member, may rotate relative to the housing during both operations. One of the first member and the second member, e.g. the second member, may rotate relative to the housing during only one of the operations, e.g. during dose setting or during dose delivery.

In one embodiment, the second member of the dose setting and/or drive mechanism is configured to be connected or is connected to the second portion of the user interface member. In one embodiment, the electronic system comprises at least one of, an arbitrarily selected plurality of, or all of the following units or components:
an electrical motion sensing unit. The motion sensing unit will be explained in more detail below.
a communication unit. The communication unit may be provided to establish the communication interface between the electronic system and another device such as an electronic device such as a portable device, e.g. a portable or non-portable computer, a mobile phone or a tablet. The communication unit may be a wireless unit, e.g. an RF communication unit, such as a Bluetooth unit. The communication unit may be provided to transmit dose data from the electronic system to the other device, e.g. information on the amount of drug delivered by the device in a delivery operation.
a memory unit. The memory unit may be provided to store executable program code and/or data on dose information which has been calculated by the electronic system, preferably dose data on the delivered dose or doses. The dose data may be determined via the motion sensing unit. From the memory unit, the data may be retrievable for transmission to another device, e.g. via the communication unit.

In one embodiment, the motion sensing unit is configured to generate one or more electrical motion signals. The motion signal(s) may be suitable to quantify the relative movement between the first member and the second member, e.g. during the dose setting operation or the dose delivery operation, e.g. to obtain dose data, such as the size of the delivered dose. The first member and/or the second member may be members of the electronic system and/or the drug delivery device, e.g. the dose setting and/or drive mechanism as discussed further above. The relative movement may be relative rotational movement. For example, the first member may rotate relative to the second member during dose delivery.

In one embodiment, the electronic system is configured such that the motion sensing unit is switched from the first state into the second state, e.g. by the electronic control unit and/or in response to the signal provided by the signaling unit in response to the movement of the first portion into the signaling position. In the first state, the motion sensing unit may be not operative to sense movement of the first member relative to the second member. In the second state, the motion sensing unit may be operative. In the second state, the motion sensing unit may have a power consumption which is greater than in the first state. The increase in power consumption of the motion sensing unit may contribute to or define the increased power consumption of the electronic system in the second state.

In one embodiment, the motion sensing unit is configured to operate during the dose delivery operation, preferably only during the dose delivery operation. The motion sensing unit may be configured to monitor the dose delivery operation, e.g. the rotation of the first member relative to the second member. Thus, from the motion signals, positional information on the relative position between the first member and the second member can be gathered. Alternatively or additionally, it is also possible to gather positional information between two members in the dose setting operation. However, in order to calculate dose information or data on the dose delivery during the dose delivery operation, it is advantageous to monitor the movements during the dose delivery operation by the motion sensing unit.

In one embodiment, the electronic system is configured such that the communication unit and/or the motion sensing unit is activated in response to the signal. The communication unit may be activated in response to a dose operation and/or in response to the activation operation, e.g. only in response to the activation operation or in response to the activation operation and the dose operation. The communication unit may be provided to perform a data transmission to a further device, e.g. a phone or a laptop, as has been discussed above. When the communication unit is activated, e.g. in response to the activation operation, the electronic system may attempt to transmit data, e.g. dose data, to the further device, which may be paired with the electronic system, or synchronize data with the further device via the communication unit. The motion sensing unit may be still non-operational in response to the activation operation, i.e. the motion sensing unit, in contrast to the communication unit may not be activated by the activation operation. The activation operation and/or the dose operation may be used to trigger a data transmission or synchronization procedure with the further device. During the dose operation, the motion sensing unit is preferably operational as well, e.g. when the dose delivery operation for delivering a previously set dose is performed. After the dose delivery operation has been completed, the communication unit may transmit or attempt to transmit the data to the further device.

In one embodiment, the electronic control unit or the electronic system is configured to calculate dose information or data utilizing the motion signals generated by the motion sensing unit. As noted previously, the dose information preferably is information on the size of the dose which is delivered in the dose delivery operation.

In one embodiment, when in the rest position, the first portion is movable relative to the second portion in two different directions, e.g. in opposite rotational directions. One of the directions may be the one towards the signaling position where the signal is generated for switching the electronic system to the state of higher power consumption, e.g. to activate the motion sensing unit and, preferably the communication unit. The other direction may be direction away from the signaling position. In this direction a signal may be generated as well, e.g. via an according switch which, when triggered, indicates rotation in that direction. However, the electronic control unit, preferably only, switches the system to a state of higher power consumption with the motion sensing unit being active, when the signaling position is detected and the according signal is generated. The other direction can be used to manually activate (only) the communication unit, e.g. to perform a pairing or synchronization operation with another device.

The operation of the user interface member associated with the activation of the communication unit may be the activation operation. A pairing or synchronization operation may be integrated into the operation sequence of the electronic system after the motion sensing unit has acquired dose data, e.g. related to the delivered dose in the dose delivery operation.

In one embodiment, the electronic system is configured such that the electronic system is switched only from the first state into the second state, when the first portion of the user interface member is held, maintained or kept away from the rest position, e.g. in the signaling position, for a time exceeding a predetermined time. The predetermined time may be 2 s, 3 s, 4 s, or 5 s. Hence, the first portion may have to be kept in the signaling position, e.g. against the bias tending to move it into the rest position, for the system to be switched into the second state. This is particularly suitable for an activation operation which is different from the dose operation, as it requires a conscious decision by the user to switch the system and as an accidental movement which maintains the first portion away from the rest position for a predetermined time is less likely to occur than accidental displacements. The first portion may have to be rotated or axially displaced away from the rest position and held away from that position for the predetermined time before the system is switched to the second state of higher power consumption.

In one embodiment, the motion sensing unit comprises one or more sensors and/or one or more emitters, e.g. one or more optoelectronic radiation sensors or detectors and/or one or more optoelectronic radiation emitters. The sensors may be configured to generate motion signal(s) in response to movement of the first member relative to the second member. The emitters may excite the sensor signals.

In one embodiment, during the dose setting operation, the dose may be set, e.g. between a minimum settable dose and a maximum settable dose. The dose may be set, preferably in quantities corresponding to whole-number multiples of one unit dosage increment.

In one embodiment, the separation, e.g. the axial separation, between the first position and the second position of the user interface member, e.g. relative to the housing, is determined by, for example equal to, a switching distance, e.g. a clutch release distance. The switching distance may be the distance by which the second member of the dose setting and drive mechanism has to be moved relative to the first member of the dose setting and drive mechanism in order to switch the dose setting and drive mechanism from a dose setting configuration of the mechanism to a dose delivery configuration of the mechanism. In the first position, the dose setting and drive mechanism may be in the dose setting configuration. In the second position, the dose setting and drive mechanism may be in the dose delivery configuration. In the dose setting configuration or the first position, for example, the members of the dose setting and drive mechanism may be rotationally locked as has been discussed further above. In the dose delivery configuration or the second position, relative rotation is allowed, e.g. the first member may rotate relative to the second member and the housing during dose delivery. During the dose delivery operation, the second member may be rotationally locked relative to the housing.

In one embodiment, the, e.g. axial, separation between the first position and the second position is greater than or equal to the distance by which the second member has to be moved, e.g. axially, relative to the first member in order to release the rotational lock. Specifically, during the movement of the user interface member from the first position to the second position, the rotational lock may be released by displacing the second member axially, e.g. distally, relative to the first member. The distance to release the rotational lock may correspond to the switching distance. The signal may be provided and/or the electronic system may be switched to the second state immediately when the member is moved from the first position into the second position.

In one embodiment, the electronic system comprises a power supply, e.g. a rechargeable or non-rechargeable battery.

In one embodiment, in the second state, the electronic system is configured to gather information or data related to the size of the currently dispensed or delivered dose during the delivery operation, e.g. via the motion sensing unit. Consequently, the motion sensing unit may be configured to contribute to retrieve dose data on the dose delivered in the delivery operation, e.g. the currently delivered dose during the dose delivery operation.

In one embodiment, in the second state, the electronic system is configured to store dose data in a dose memory or memory unit of the electronic system. The memory may be transitory or non-transitory. The dose data is expediently derived using measurements or signals of the motion sensing unit.

In one embodiment, in the second state, the electronic system is configured to transmit dose data, e.g. dose data retrieved from the memory, by means of the communication unit to another device or system such as a computing device, e.g. a mobile phone or a portable or non-portable computing unit.

In one embodiment, the electronic system comprises one user interface member, e.g. one integral member, for the dose setting operation and the dose delivery operation or two different user interface members, where one of these members is the user interface member for dose setting and the other one is the user interface member for dose delivery. The two different members are expediently movable relative to one another, e.g. to switch between a dose setting configuration and a dose delivery configuration. If one interface member is used for dose setting and dose delivery, this interface member may have the setting surface and the delivery surface, which, preferably, are not movable relative to one another, especially not for or during dose delivery and/or not for or during dose setting. If two different user-interface members are used, the setting surface and the delivery surface may be on different members and movable relative to one another for or during dose delivery and/or for or during dose setting.

In one embodiment, the electronic system comprises a timer unit. The timer unit may be configured to deactivate the motion sensing unit and/or other electrically powered units of the electronic system after a predetermined time period has elapsed and, preferably when in this time period no motion signal is generated. The timer unit may trigger or cause the electronic system to be switched from the second state back to the first state. In other words, the electronic system may be configured to switch from the second state back to the first state, preferably when for a predetermined time no motion signal is generated and/or received by the electronic control unit.

In one embodiment, the user interface member is a dose setting and/or an injection button of or for the drug delivery device.

In one embodiment, the dose setting operation and/or the dose delivery operation requires movement of the second portion of the user interface member, e.g. relative to the housing.

In one embodiment, the electronic system comprises a feedback unit. The feedback unit may be configured to generate a feedback perceivable by the user. The feedback may enable the user to determine whether the system is in the first state or in the second state. Preferably, in the first state, there is no perceivable feedback provided and the feedback is indicative for the second state. The feedback may be a feedback signal, such as an optical signal, for example. The feedback signal may be provided by a light source, such as a light-emitting diode. The light source may operate in a pulsed or flashing manner for providing the feedback.

In one embodiment, the device is a manually driven device, e.g. user driven.

In one embodiment, the drug delivery device comprises a reservoir retainer for retaining a reservoir with drug, e.g. a cartridge, and/or the device comprises the reservoir with drug. The reservoir may comprise drug sufficient for a plurality of, preferably user-settable, doses to be delivered by the drug delivery device.

In one embodiment, the drug delivery device is a pen-type device.

In one embodiment, the electronic system is configured as a, preferably reusable, add-on for a drug delivery device unit. The system may be configured to be attached to the drug delivery device unit. That is to say, the electronic system may be configured to be used with a plurality of drug delivery device units. The respective drug delivery device unit may be a disposable drug delivery device unit and/or the respective drug delivery device unit may be fully operational for performing dose setting operations and dose delivery operations. The drug delivery device unit may comprise the reservoir.

In one embodiment, the power supply is non-replaceable.

In one embodiment, a kit for a drug delivery device comprises the drug delivery device unit and the electronic system. The system may be attachable to the device unit to form the drug delivery device. Features disclosed above and below for the drug delivery device, especially the ones that are not directly related to the electronic system, should also apply for the drug delivery device unit and vice versa.

“Distal” is used herein to specify directions, ends or surfaces which are arranged or are to be arranged to face or point towards a dispensing end of the drug delivery device or components thereof and/or point away from, are to be arranged to face away from or face away from the proximal end. On the other hand, “proximal” is used to specify directions, ends or surfaces which are arranged or are to be arranged to face away from or point away from the dispensing end and/or from the distal end of the drug delivery device or components thereof. The distal end may be the end closest to the dispensing and/or furthest away from the proximal end and the proximal end may be the end furthest away from the dispensing end. A proximal surface may face away from the distal end and/or towards the proximal end. A distal surface may face towards the distal end and/or away from the proximal end. The dispensing end may be the needle end, where a needle unit is or is to be mounted to the device, for example.

In a particularly advantageous embodiment, an electronic system for a drug delivery device comprises:
at least one user interface member configured to be manipulated by a user for performing a dose operation, e.g. a dose setting operation to set a dose of drug to be delivered by the drug delivery device and/or a dose delivery operation for delivering a set dose,
an electronic control unit, the electronic control unit being configured to control operation of the electronic system, the electronic system having a first state and a second state, wherein the electronic system has an increased electrical power consumption in the second state as compared to the first state,
an electrical signaling unit, the signaling unit being configured to provide an electrical signal indicative that the user interface member is being manipulated, wherein
the electronic control unit is configured to switch the electronic system from the first state into the second state in response to the electrical signal, and wherein
the user interface member comprises a first portion and a second portion, wherein the first portion is movable relative to the second portion from a rest position of the first portion to a signaling position of the first portion, wherein the signaling unit is configured to provide the electrical signal in response to the movement of the first portion relative to the second portion from the rest position into the signaling position.

Features, which are disclosed in conjunction with different aspects and embodiments may be combined with one another even if such a combination is not explicitly discussed above or below. Further aspects, embodiments and advantages will become apparent from the following description of the exemplary embodiments in conjunction with the drawings.

### BRIEF DESCRIPTION OF THE FIGURES

FIG. 1 illustrates an embodiment of a drug delivery device or drug delivery device unit.

FIG. 2 illustrates schematically an electronic system for a drug delivery device, e.g. the one in FIG. 1.

FIG. 3 illustrates schematically an embodiment of an electronic system for a drug delivery device, e.g. the one in FIG. 1.

FIGS. 4A to 4H schematically illustrate an embodiment of an electronic system for a drug delivery device, e.g. the one in FIG. 1.

FIG. 5 illustrates another embodiment of an electronic system for a drug delivery device.

FIGS. 6A and 6B illustrate another embodiment of an electronic system for a drug delivery device.

FIGS. 7A through 7C illustrate another embodiment of an electronic system for a drug delivery device.

### DETAILED DESCRIPTION

In the drawings identical features, features of the same kind or identically or similarly acting features may be provided with the same reference numerals.

In the following, some concepts will be described with reference to an insulin injection device. The systems described herein may be implemented in this device or used as an add-on module to the device. The present disclosure is however not limited to such an application and may equally well be used for or in injection devices that are configured to eject other medicaments or drug delivery devices in general, preferably pen-type devices and/or injection devices.

In the following, embodiments are provided in relation to injection devices, in particular to variable dose injection devices, which record and/or track data on doses delivered thereby. These data may include the size of the selected dose and/or the size of the actually delivered dose, the time and date of administration, the duration of the administration and the like. Features described herein may include power management techniques (e.g. to facilitate small batteries and/or to enable efficient power usage).

Certain embodiments in this document are illustrated with respect to an injection device where an injection button and grip (dose setting member or dose setter) are combined e.g. similar to Sanofi's ALLSTAR® device. The injection button may provide the user interface member for initiating and/or performing a dose delivery operation of the drug delivery device. The grip or knob may provide the user interface member for initiating and/or performing a dose setting operation. The devices may be of the dial extension type, i.e. their length increases during dose setting. Other injection devices with the same kinematical behavior of the dial extension and button during dose setting and dose expelling operational mode are known as, for example, the Kwikpen® or Savvio® device marketed by Eli Lilly and the FlexPen®, or Novopen® device marketed by Novo Nordisk. An application of the general principles to these devices therefore appears straightforward and further explanations will be omitted. However, the general principles of the present disclosure are not limited to that kinematical behavior. Certain other embodiments may be conceived for application to injection devices where there are separate injection button and grip components/dose setting members e.g. Sanofi's SoloSTAR®. Thus, the present disclosure also relates to systems with two separate user interface members, one for the dose setting operation and one for the dose delivery operation. In order to switch between a dose setting configuration of the device and a dose delivery configuration, the user interface member for dose delivery may be moved relative to the user interface member for dose setting. If one user interface member is provided, the user interface member may be moved distally relative to a housing. In the course of the respective movement, a clutch between two members of the dose setting and drive mechanism of the device changes its state, e.g. from engaged to released or vice versa. When the clutch, e.g. formed by sets of meshing teeth on the two members, is engaged, the two members may be rotationally locked to one another and when the clutch is disengaged or released, one of the members may be permitted to rotate relative to the other one of the two members. One of the members may be a drive member or drive sleeve which engages a piston rod of the dose setting and drive mechanism. The drive sleeve may be designed to rotate relative to the housing during dose setting and may be rotationally locked relative to the housing during dose delivery. The engagement between drive sleeve and piston rod may be a threaded engagement. Thus, as the drive sleeve cannot rotate during dose delivery, axial movement of the drive sleeve relative to the housing will cause the piston rod to rotate. This rotation may be converted into axial displacement of the piston rod during the delivery operation by a threaded coupling between piston rod and housing.

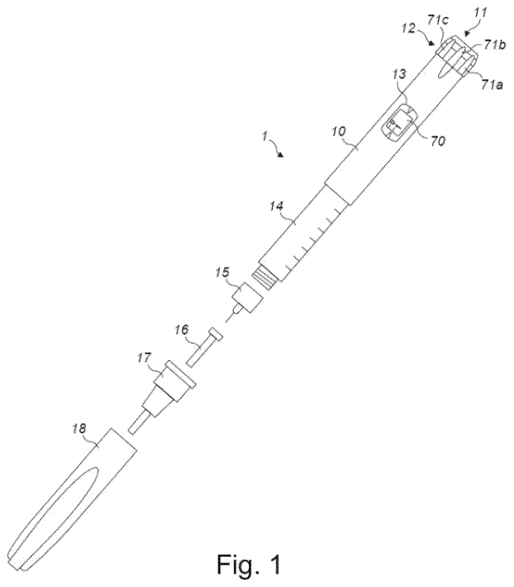

The injection device 1 of FIG. 1 is an injection pen that comprises a housing 10 and contains a container 14, e.g. an insulin container, or a receptacle for such a container. The container may contain a drug, e.g. insulin. The container may be a cartridge or a receptacle for a cartridge which may contain the cartridge or be configured to receive the cartridge. A needle 15 can be affixed to the container or the receptacle. The container may be a cartridge and the receptacle may be a cartridge holder. The needle is protected by an inner needle cap 16 and either an outer needle cap 17 or another cap 18. An insulin dose to be ejected from injection device 1 can be set, programmed, or ‘dialled in’ by turning a dosage knob 12, and a currently programmed or set dose is then displayed via dosage window 13, for instance in multiples of units. The units may be determined by the dose setting mechanism which may permit relative rotation of the knob 12 to the housing 10 only in whole-number multiples of one unit setting increment, which may define one dosage increment. This may be achieved by an appropriate ratchet system, for example. The indicia displayed in the window may be provided on a number sleeve or dial sleeve 70. For example, where the injection device 1 is configured to administer human insulin, the dosage may be displayed in so-called International Units (IU), wherein one IU is the biological equivalent of about 45.5 micrograms of pure crystalline insulin (1/22 mg). Other units may be employed in injection devices for delivering analogue insulin or other medicaments. It should be noted that the selected dose may equally well be displayed differently than as shown in the dosage window 13 in FIG. 1.

The dosage window 13 may be in the form of an aperture in the housing 10 or a transparent separate component inserted into an aperture of the housing, where the separate component may incorporate a magnifying lens. The dosage window 13 permits a user to view a limited portion of a dial sleeve 70 that is configured to move when the dosage knob 12 is turned, to provide a visual indication of a currently programmed dose. The dosage knob 12 is rotated on a helical path with respect to the housing 10 when turned during programming.

In this example, the dosage knob 12 includes one or more formations 71a, 71b, 71c to facilitate attachment of a data collection device or electronic system. An electronic system which may be attachable to the user interface member (knob 12 and/or button 11) or, in general, to elements or members of a dose setting and drive mechanism of the drug delivery device 1 will be described in more detail below. The electronic system may be provided within the user interface member, for example. The electronic system which will be described in more detail below can also be configured as an add-on for a drug delivery device.

The injection device 1 may be configured so that turning the dosage knob 12 causes a mechanical click sound to provide acoustical feedback to a user. In this embodiment, the dosage knob or dose button 12 also acts as an injection button 11. When needle 15 is stuck into a skin portion of a patient, and then dosage knob 12/injection button 11 is pushed in an axial direction, the insulin dose displayed in display or dosage window 13 will be ejected from injection device 1. When the needle 15 of injection device 1 remains for a certain time in the skin portion after the dosage knob 12 is pushed home, the dose is injected into the patient's body. Ejection of the insulin dose may also cause a mechanical click sound, which is however different from the sounds produced when rotating the dosage knob 12 during dialing of the dose.

In this embodiment, during delivery of the insulin dose, the dosage knob 12 is returned to its initial position in an axial movement, without rotation, while the dial sleeve 70 or number sleeve 70 is rotated to return to its initial position, e.g. to display a dose of zero units. As noted already, the disclosure is not restricted to insulin but should encompass all drugs in the drug container 14, especially liquid drugs or drug formulations.

Injection device 1 may be used for several injection processes until either the insulin container 14 is empty or the expiration date of the medicament in the injection device 1 (e.g. 28 days after the first use) is reached.

Furthermore, before using injection device 1 for the first time, it may be necessary to perform a so-called “prime shot” to ensure fluid is flowing correctly from insulin container 14 and needle 15, for instance by selecting two units of insulin and pressing dosage knob 12 while holding injection device 1 with the needle 15 upwards. For simplicity of presentation, in the following, it will be assumed that the ejected amounts substantially correspond to the injected doses, so that, for instance the amount of medicament ejected from the injection device 1 is equal to the dose received by the user.

As explained above, the dosage knob 12 also functions as an injection button 11 so that the same component is used for dialling/setting the dose and dispensing/delivering the dose. Again, we note that a configuration with two different user interface members which, preferably only in a limited fashion, are movable relative to one another is also possible. The following discussion will, however, focus on a single user interface member which provides dose setting and dose delivery functionality. In other words, a setting surface of the member which is touched by the user for the dose setting operation and a dose delivery surface which is touched by the user for the dose delivery operation are immovably connected. Alternatively, they may be movable relative to one another, in case different user interface members are used. During the respective operation, the user interface member is preferably moved relative to the body or housing of the device. During dose setting the user interface member is moved proximally and/or rotates relative to the housing. During dose delivery, the user interface member moves axially, e.g. distally, preferably without rotating relative to the housing or body.

In the following, a general setup for an electronic system for a drug delivery device is disclosed.

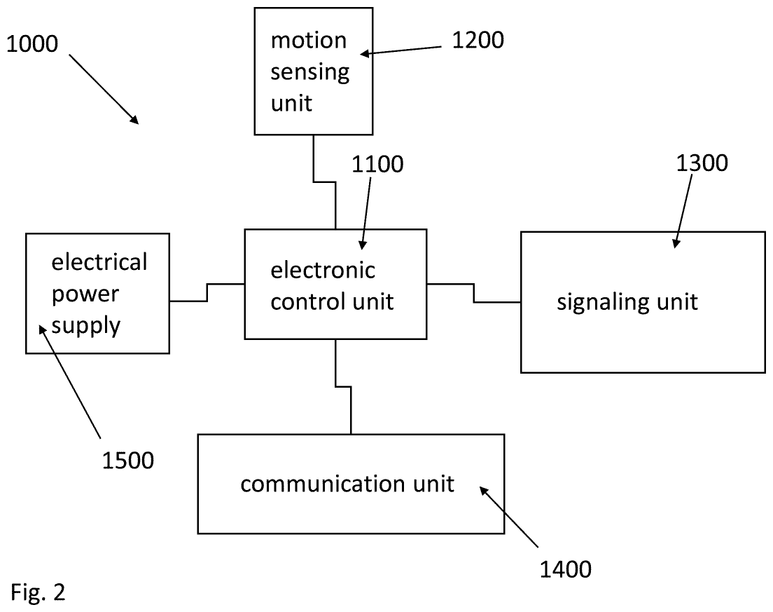

FIG. 2 illustrates a general configuration of elements of an electronic system 1000 which can be used in or for a drug delivery device, for example the device or device unit discussed further above or in different devices.

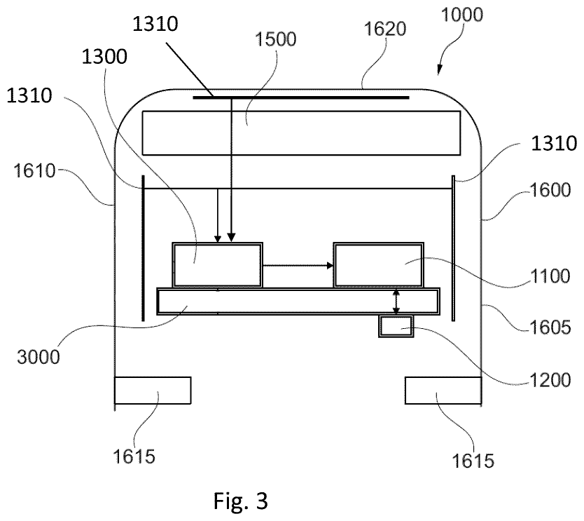

The electronic system 1000 comprises an electronic control unit 1100. The control unit may comprise a processor, e.g. a microcontroller or an ASIC. Also, the control unit 1100 may comprise one, or a plurality of memory units, such as a program memory and/or a main memory. The program memory may be designed to store program code which when carried out by the system controls operation of the system and/or the electronic control unit. The control unit 1100 is expediently designed to control operation of the electronic system 1000. The control unit 1100 may communicate via wired interfaces or wireless interfaces with further units of the electronic system 1000. The control unit 1100 may transmit signals containing commands and/or data to the respective unit and/or receive signals and/or data from the respective unit. The connections between the units and the electronic control unit 1100 are symbolized by the lines in FIG. 2. However, there also may be connections between the units, which are not illustrated explicitly. The control unit 1100 may be arranged on a conductor carrier, e.g. a (printed) circuit board (see reference 3000 in FIG. 3). The other unit(s) of the electronic system may comprise one or more components which are arranged on the conductor carrier as well.

Electronic system 1000 further comprises an electrical motion sensing unit 1200. The motion sensing unit 1200 may comprise one sensor e.g. only one sensor, or a plurality of sensors. The motion sensing unit is expediently designed to generate motion signals, such as electrical signals, which are indicative for movement of one member of the electronic system or the drug delivery device relative to another member—e.g. movement of the dial sleeve or number sleeve relative to the drive sleeve or button/knob in the device discussed further above—, where the sensor may be fixedly connected to one of the members, e.g. the knob or button. The relative movement expediently occurs during the dose delivery operation. The respective sensor may be an optoelectronic sensor. The optoelectronic sensor may sense radiation emerging from a member moving relative to the sensor and impinging on the sensor to excite sensor signals or motion signals in the sensor, e.g. an optical encoder component. The radiation may be radiation reflected by the member and impinging on the member from a radiation source, such as an optoelectronic radiation source, e.g. an LED. The radiation source may be an IR source (IR-LED, an InfraRed Light Emitting Diode). The radiation source may be part of the sensor arrangement comprising the at least one sensor. One possible embodiment of the sensor is an IR-sensor which is configured to detect infrared light. The light source and the sensor may be arranged on the same component or member. The general functionality of optoelectronic sensor arrangements suitable for the electronic system discussed herein is disclosed in WO 2019/101962 A1, where the entire disclosure content is explicitly incorporated herein by reference for all purposes, especially as regards the different sensor arrangements and configurations. However, it should be noted that other sensor arrangements, e.g. using magnetic sensors, could be employed as well. In a motion sensing unit which has an electrically operated sensor and/or an electrically operated source for stimulating the sensor—such as a radiation emitter and an associated sensor—the power consumption may be particularly high and, hence, appropriate power management of electrical power available for powering the system may have a particular impact. The motion sensing unit 1200 may be designed to detect and preferably measure or quantify relative movement of one member of a dose setting and drive mechanism of or for the drug delivery device relative to another member of the dose setting and drive mechanism or relative to the housing 10 during a dose delivery operation. For example, the motion sensing unit may measure or detect relative rotational movement of two movable members of the dose setting and drive mechanism with respect to one another. Based on movement data received from or calculated from the signals of the unit 1200, the electronic system, e.g. the control unit, may calculate dose data, e.g. data on the currently delivered dose. The motion sensing unit 1200 is expediently configured to quantify the relative movement between a first member and a second member of the electronic system or the drug delivery device. The relative movement may be indicative for the delivered dose. The relative movement may be relative rotational movement. For example, the first member may rotate relative to the second member, such as during dose delivery. The motion sensing unit is expediently suitable to quantify the relative movement in whole-number multiples of one unit setting increment angle. The unit setting increment may be or may be defined by an angle greater than or equal to one of the following values: 5°, 10°. The unit setting increment may be or may be defined by an angle less than or equal to one of the following values: 25°, 20°. The unit setting increment may be between 5° and 25°, for example. The unit setting increment may correspond to a relative rotation of 15°, for example. The unit setting increment angle may be the rotation required to set the smallest settable dose to be delivered by the device. The increment may be defined by a ratchet system for example. As has been explained above, the amount or distance of the relative (rotational) movement determined by the motion sensing unit between the first and second members is characteristic for the currently set dose in a dose setting operation or for the currently dispensed dose in a dose delivery operation. The size of the dose delivered may be determined by or correspond to the distance by which a piston rod of the dose setting and drive mechanism is displaced distally relative to the housing during the dose delivery operation.

The electronic system 1000 further comprises a signaling unit 1300. The signaling unit may be associated with the user interface member or members (knob 12 or button 11 in the device discussed above). Via the signaling unit 1300 the manipulation of the member for setting and/or for delivering a dose may be detected. The signaling unit is configured to generate an electrical signal in response to a manipulation of the user interface member, preferably in direct response to a manipulation, e.g. a unidirectional movement. The user interface member may have a setting surface which is arranged to be touched by the user for performing the dose setting operation and/or a delivery surface which is arranged to be touched by the user for performing the dose delivery operation. The setting surface may face in the radial direction and the delivery surface may face in the axial, e.g. proximal, direction. Signal generation may require movement of at least a portion of the user interface member, e.g. relative to another portion of the user interface member and/or the housing. This will be explained in more detail further below. The element generating the signal may be an electrical sensor or switch, such as a micro force switch, for example. The signals generated by the signaling unit in response to manipulations may allow to distinguish between different directions into which the user interface member is moved and/or different surfaces of the user interface member which are manipulated by the user. In this case, a plurality of switches may be provided. The signaling unit is expediently configured such that the electrical signal(s) it is configured to generate responsive to a manipulation allows gathering information on what manipulation is currently being performed, is intended to be performed or has been performed, e.g. a dose setting operation, a dose delivery operation or a different operation. The signal generated by the signaling unit 1300 may be an activation prompt signal or use signal. The signaling unit 1300 is operatively connected to the electronic control unit 1100, for example. The signal provided by the signaling unit may be received and/or processed by the electronic control unit 1100.

The electronic system 1000 further comprises a communication unit 1400, e.g. an RF, WiFi and/or Bluetooth unit. The communication unit may be provided as a communication interface between the system or the drug delivery device and an external device, such as other electronic devices, e.g. mobile phones, personal computers, laptops and so on. For example, dose data may be transmitted by the communication unit to the external device and/or synchronized with the device. The dose data may be used for a dose log or dose history established in the external device. The communication unit may be provided for wireless communication.

Occurrence of the signal generated by the signaling unit 1300 may, preferably immediately or directly, cause the electronic control unit 1100 to switch the electronic system from a first state or rest state (e.g. the state the system has when it is not needed, e.g. in a dormant state, where the rest state is optimized in terms of power consumption) to a second state of higher power consumption, e.g. by activating the motion sensing unit 1200 and/or the communication unit 1400. A single signal generated by the signaling unit may be sufficient to trigger the switching to the second state. For switching the system to the second state, the control unit 1100 may send an activation signal to the respective unit. In the second state, the motion sensing unit and/or the communication unit may be operable. In the first state, the motion sensing unit and/or the communication unit, preferably, cannot be operated. In this way, the functionality of the electrically operated units may be available when needed. The power consumption required in the first state for the signaling unit being operational advantageously is smaller than the power consumption when the communication unit and/or the motion sensing unit are/is operable. The activation prompt or use signal may be generated responsive to a manipulation of the user interface member or at least a portion thereof. The manipulation may involve only unidirectional movement. The manipulation may involve only rotational or only axial movement of the user interface member or the portion. The activation operation of the user interface member may be different from the dose setting operation and from the dose delivery operation. For example, the activation operation may involve an attempted rotation of a portion of the user interface member counter to a dose setting direction, e.g. in a zero dose position of the user interface member. Thus, a dedicated activation operation may be required to cause the system to switch to the second state. Hence, the user can decide whether the electronic functionality is required or, e.g. in view of urgency or other reasons, opt that the drug delivery device should be used without the electronic functionalities, which is preferably possible. Alternatively, the signal generation may be integrated into the dose setting operation or the dose delivery operation, e.g. by using a relative movement between different portions of the user interface member. Such a relative movement, which is preferably limited, expediently cannot be detected by the user as being required for activating the electronic system.

The electronic system 1000 further comprises an electrical power supply 1500, such as a rechargeable or non-rechargeable battery. The power supply 1500 may provide electrical power to the respective units of the electronic system.

In one embodiment, the power consumption, in particular the maximum power consumption, of the electronic system in the first state, e.g. prior to generation of the use or activation prompt signal, may be less than or equal to one of the following values: 300 nA, 250 nA, 200 nA (nA: nanoampere). Alternatively or additionally, in the second state of the electronic system, the power consumption, in particular the minimum power consumption, may be greater than or equal to one of the following values: 0.5 mA, 0.6 mA, 0.8 mA (mA: milliampere). The difference can result from the power consumption of the motion sensing unit 1200 and/or of the communication unit 1400 which may be active or operable in the second state and switched off or in a sleep state in the first state of the electronic system 1000.

In one embodiment, the power consumption P2, e.g. the minimum or maximum power consumption, in the second state may be greater than or equal to at least one of the following values: 2*P1, 3*P1, 4*P1, 5*P1, 10*P1, 20*P1, 30*P1, 40*P1, 50*P1, 100*P1, 500*P1, 1000*P1, 2000*P1, 5000*P1, 10000*P1 where P1 is the power consumption in the first state. In the second state, the motion sensing unit may be active and/or the communication unit may be active, e.g. for wireless communication.

When the system is in the first state, e.g. with neither the motion sensing unit being active nor the communication unit, the current consumption may be 200 nA. When (only) the motion sensing unit is active, the power consumption may be 0.85 mA. When the communication unit is active, e.g. in addition to the motion sensing unit or only the communication unit, the power consumption may be 1.85 mA.

Although not explicitly depicted, the electronic system preferably comprises a, e.g. permanent and/or non-volatile, storage or memory unit, which may store data related to the operation of the drug delivery device such as dose (history) data, for example.

In one embodiment, the electronic control unit 1100 is configured to reduce the power consumption of the respective unit, i.e. to switch the unit back to the first state. This is suitable, for example, if an event which is relevant for that unit, e.g. a motion sensing event (motion signal) for the motion sensing unit, has not occurred in a predetermined time interval after the unit has been switched from the first state into the second state and/or after the use signal has been generated. The monitoring of the time interval may be achieved by a timer unit which is operatively connected to the electronic control unit (not explicitly shown). In case, after the use or activation prompt signal, there is no signal generated by the motion sensing unit within the predetermined time interval, the entire system may be switched to the first state again. This time interval may be greater than or equal to one of the following values 5 s, 10 s, 15 s, 20 s, 25 s, 30 s. Alternatively or additionally the time interval may be less than or equal to one of the following values: 180 s, 150 s, 120 s, 90 s, 80 s, 70 s, 60 s, 50 s, 45 s, 40 s, 35 s, 30 s. The time interval may be between 5 and 180 seconds, e.g. 30 s or 180 s. The entire system may be switched back to the first state in case no motion signal is generated within the predetermined time interval. The predetermined time interval is expediently constant.

In one embodiment, the electronic system comprises a feedback unit (not explicitly shown). The feedback unit is configured to generate a feedback perceivable by the user. The feedback can enable the user to determine whether the system is in the first state or in the second state. Preferably, in the first state, there is no perceivable feedback provided and/or the feedback is indicative for the second state. The feedback may be a feedback signal, such as an optical signal, for example. The feedback signal may be provided by a light source, such as a light-emitting diode. The light source may operate in a pulsed or flashing manner for providing the feedback.

The respective unit which has been described above may be integrated into the user interface member of the electronic system which is discussed in further detail below in conjunction with various embodiments.

It goes without saying that the electronic system 1000 may comprise further electronic units other than the ones shown such as other sensing units, which sense or detect different quantities or events than the relative movements which the motion sensing unit detects.

In the following some more detailed embodiments of the electronic system are described. It should be noted that features which have been discussed above do also apply for these embodiments.

FIG. 3 schematically illustrates an embodiment of an electronic system 1000. The system 1000 comprises a user interface member 1600. The user interface member is designed to be operated during a dose setting operation and/or a dose delivery operation by the user. The user interface member 1600 has different exterior operation surfaces. The operation surfaces may be defined by exterior surfaces which are accessible from the exterior of a user interface member housing or body 1605, preferably when the user interface member is connected to a drug delivery device unit or integrated into a device such as the unit or the device discussed in conjunction with FIG. 1. The user interface member 1600 has a setting surface 1610 which is arranged to be gripped by the user for dose setting, e.g. with two fingers such as the index finger and the thumb. The setting surface is a radially facing surface, which, preferably circumferentially, delimits the user interface member 1600 with respect to the exterior. The user interface member 1600 also has a delivery surface 1620. The delivery surface is arranged to be contacted, e.g. pressed and/or moved distally, by the user for dose delivery. The delivery surface 1620 is an axially oriented surface, e.g. a proximally facing surface. As noted above, embodiments of the disclosure can employ different user interface members for setting and delivery.

Within the user interface member 1600, e.g. within an interior hollow defined by the user interface member body 1605, some additional elements or units of the electronic system are housed. Specifically, the electronic system comprises the electronic control unit 1100. The system also comprises a conductor carrier 3000, e.g. a circuit board such as a printed circuit board. Conductors on the conductor carrier may conductively connect the electronic control unit to further electrical or electronic units or members of the system. The electronic control unit is arranged on the conductor carrier, e.g. mounted to the carrier.

The electronic system 1000 comprises the signaling unit 1300. In the depicted embodiment, the signaling unit has at least one sensor or switch or a plurality of sensors or switches 1310. In the depicted embodiment, at least one switch 1310 is associated with the setting surface 1610. Alternatively or additionally, at least one switch 1310 is associated with the delivery surface 1620. The respective switch is expediently configured to generate an electrical signal or switch signal when the user interface member or a portion thereof is moved for performing the dose setting operation (the sensor or switch is expediently associated with the setting surface) or the dose delivery operation (the sensor or switch is expediently associated with the delivery surface). For example, for the dose setting operation, the user interface member 1600 may be rotated relative to the housing 10. A plurality of switches may be provided, e.g. to enable the control unit to distinguish between rotations in different directions. For the dose delivery operation, the user interface member can be moved axially towards the housing, e.g. to switch the clutch such as from the state where the dial sleeve and the drive member are rotationally locked for dose setting to a state where relative rotation is allowed for dose delivery. The user interface member is preferably biased, e.g. by a clutch spring (not shown), to the position it has for dose setting which may be proximally offset to the one for dose delivery by the clutch switching distance. The clutch switching distance (the distance the user interface member has to be moved in order to switch the clutch) is for example greater than or equal to 1.5 mm. The electrical signal generated by the signaling unit—use or activation prompt signal—may directly trigger the electronic control unit 1100 to switch the system from the first state to the second state. The movement for the manipulation which triggers generation of the signal is expediently unidirectional, i.e. only movement in one direction is required. In this way complicated manipulations of the user interface member 1600 for switching the system to the second state can be avoided.

The system furthermore comprises the motion sensing unit 1200 which is only schematically represented and, preferably, comprises one or more optoelectronic sensors and/one or more associated radiation emitters, e.g. IR sensors and IR emitters. The motion sensing unit may be bidirectionally conductively connected to the electronic control unit 1100 as hinted by the double arrow. One direction may be the one where the activation signal is transmitted from the electronic control unit to the motion sensing unit. In the other direction, motion signals may be sent from the motion sensing unit to the control unit, which may process the signals further, e.g. to calculate dose information or data. The motion sensing unit 1200 may be arranged on that side of the conductor carrier 3000 which faces away from the control unit 1100.

Further, the system 1000 comprises the power supply 1500, e.g. a battery, such as a coin cell. The power supply may be configured to provide a total charge of approx. 25-500 mAh at a voltage of approx. 1.4-3V. This may be achieved or assisted by stacking multiple coin cells, for example. The power supply 1500 is conductively connected or connectable to the other components of the electronic system, which require electrical power for operating. The conductive connection is not explicitly illustrated in FIG. 3. A metal pressing may be provided for connecting the power supply 1500 to the conductor carrier 3000 which may distribute the power to further elements via conductors on the carrier. The power supply may, however, be arranged so as to extend along one main surface of the conductor carrier 3000 as depicted. The power supply, in the depicted embodiment, is arranged between the conductor carrier 3000 and the delivery surface 1620. This facilitates a compact formation of the user interface member 1600.

A radial width or diameter of the user interface member 1600 as seen from the exterior of the member, e.g. in top view onto the delivery surface, may be less than or equal to one of the following values: 2 cm, 1.5 cm. Alternatively or additionally, the radial width or diameter of the user interface member may be greater than or equal to one of the following values: 0.5 cm, 0.7 cm. The radial extension may be determined relative to the rotation axis of the user interface member during dose setting or relative to the main longitudinal axis of the user interface member, which axes may coincide. The length or axial extension of the user interface member 1600 may be less than or equal to one of the following values: 2.5 cm, 2 cm, 1.5 cm. Alternatively or additionally, the length or axial extension of the user interface member 1600 may be greater than or equal to one of the following values: 0.5 cm, 0.7 cm.

Electronic system 1000 is configured to be connected, preferably releasably, to a drug delivery device unit as an add-on unit or module. The drug delivery device unit may be electronic free. Accordingly, all electronics may be provided in the electronic system. The drug delivery device unit may be disposable. That is to say, the unit can be disposed of after a reservoir of the unit has been emptied using the drug delivery device comprising the unit and the system 1000. The electronic system 1000 could be reused for another drug delivery device unit. The drug delivery device unit is preferably configured as fully functional on its own, i.e. it could be operated for setting a dose to be delivered and deliver the set dose. One exemplary unit is the one depicted in FIG. 1. The electronic system may be a pure add-on to an, otherwise, fully functional unit, which is expediently free of electronical or electrically operated parts. Alternatively, a drug delivery device may comprise the electronic system as an integral part, i.e. a part which is disposed of together with the remainder of the device and/or necessary such that the device can be operated for setting and delivering a dose of drug, e.g. because without the electronic system the drug delivery device unit would lack a surface accessible for the user for conducting a dose setting operation or a dose delivery operation. For a connection to the drug delivery device unit, the electronic system may comprise one or more connection features 1615, e.g. snap features. The respective connection feature is arranged in a distal portion of the user interface member 1600, e.g. in the interior of the member.

The system 1000 is expediently configured to be mechanically connected, either permanently or removably/releasably, to a member of the drug delivery device unit such as a member of the dose setting and drive mechanism, e.g. to the drive sleeve or the dose knob and/or the injection button of the unit discussed in conjunction with FIG. 1. The system, e.g. via the user interface member body 1605, may be rotationally and axially locked to the member of the drug delivery device unit. The member to which the system is connected may be movable relative to the housing 10 during dose setting and/or dose delivery, e.g. rotationally and/or axially during setting and, e.g. only, axially during delivery. The member can engage the piston rod, e.g. threadedly. The dose knob and the drive sleeve of the unit in FIG. 1 may be formed integral or act as a single member during dose setting and dose delivery. During dose setting, the drive sleeve may be selectively rotationally locked to a dial sleeve of the dose setting and drive mechanism such that the dial sleeve and the drive sleeve co-rotate during dose setting, e.g. by a clutch, and the dial sleeve rotates relative to the drive sleeve during dose delivery. The dial sleeve may be the number sleeve. The relative rotation between dial sleeve and drive sleeve during dose delivery may be measured by the motion sensing unit. However, it will be readily apparent to those skilled in the art that the disclosed concepts will also work with dose setting and drive mechanisms having different ways of operation and/or different configurations.

The following embodiments illustrate implementations of the signaling unit 1300. In each case, the signaling unit is configured to provide or to generate the signal, e.g. the use signal or the activation prompt signal, in response to a movement of a first portion of the user interface member 1600 relative to a second portion of the user interface member. The first portion and the second portion are expediently connected or fixed to one another to operate as one member aside from the movement required to generate the signal and, preferably, the movement to re-establish the original relative position between the two portions. For the signal generation a limited relative mobility between the portions of the user interface member is utilized.

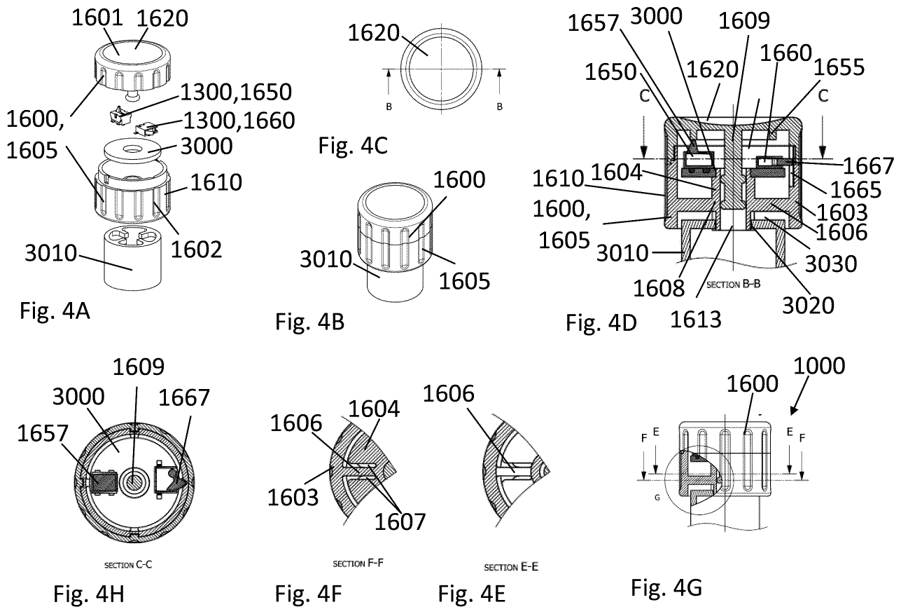

FIG. 4 illustrates one embodiment of an electronic system 1000 on the basis of a variety of schematic representations in different views as shown in FIGS. 4A through 4H. In general, the system corresponds to the one which has been described previously. Accordingly, features which has been discussed previously also apply for this embodiment unless stated otherwise or contradictory.

The electronic system 1000 pursuant to this embodiment comprises a signaling unit 1300. The signaling unit 1300 comprises a setting signaling unit. The setting signaling unit is configured to provide the signal which is indicative for a dose setting operation being initiated or performed by the user using the user interface member 1600. The setting signaling unit comprises a setting switch 1660. The setting switch 1660 is associated with the setting surface 1610 of the user interface member 1600. Alternatively or additionally to the setting signaling unit, the signaling unit comprises a delivery signaling unit. The delivery signaling unit comprises a delivery switch 1650. The delivery switch 1650 is associated with the delivery surface 1620 of the user interface member 1600. The delivery signaling unit is provided to provide a signal in response to a manipulation of the delivery surface. The setting signaling unit is provided to provide a signal in response to a manipulation of the setting surface. Via the setting signaling unit, a dose setting operation may be detected. Via the delivery signaling unit a dose delivery operation may be detected. If only one of the delivery or setting signaling unit is provided, only one of the setting on the delivery operation may be detected, whereas if both of the units are provided, the setting operation as well as the delivery operation may be detected. The electronic system may be configured to operate differently in response to a signal indicating a setting operation (setting signal) and in response to a signal indicating a delivery operation (delivery signal). The respective switch 1650, 1660 may be an electrical switch, e.g. a micro switch, such as a force switch. An additional setting switch (not shown) may be provided which enables the system to distinguish between the different rotational directions.

FIG. 4A shows on the basis of an explosive view some parts of the user interface member 1600. We note that FIG. 4 does not necessarily show all components or parts of the electronic system which have been discussed previously and will be discussed later on but only those parts which are most relevant in relation to the discussed concept.

The user interface member body 1605, in the depicted embodiment, comprises a first part 1601 and a second part 1602. During operation the two parts are expediently rigidly connected such that they act as a single member during the dose setting operation and the dose delivery operation, at least as seen from the exterior. The exterior surfaces of the user interface member may be rigid. That is to say, a section of exterior surface formed by the first part and a section of the exterior surface formed by the second part may be rotationally and axially secured to each other, e.g. by welding, such that no relative movement is possible between the sections. The first part 1601 may provide the delivery surface 1620. The second part 1602 may provide the setting surface 1610 or at least the majority thereof. As depicted, the first part 1601 may provide the delivery surface 1620 and/or a regions of the setting surface 1610 which is radially oriented and circumferentially disposed. The multiple parts of the user interface member 1600 may be provided for manufacturing reasons. A unitary construction of the user interface member body 1605 is also possible.

The conductor carrier 3000, again, is arranged within the user interface member body 1605. The conductor carrier 3000 may be rigidly connected to the user interface member. The electronic control unit 1100 is not shown in FIG. 4 for illustration purposes but may nevertheless also be arranged in the user interface member 1600, particularly on the conductor carrier 3000, as may the remaining components or units described further above.

The arrangement depicted in FIG. 4 further comprises a member 3010. The user interface member body 1605 may be fixed to the member 3010, e.g. permanently or releasable. The member 3010 may be a sleeve. Member 3010, for example, is a member of the drug delivery device unit to which the electronic system is securable as an add-on module or of the drug delivery device of which electronic system is part. The member 3010 may be the drive sleeve or dose knob of the dose setting and drive mechanism for example. The member 3010 may be rigidly connected, e.g. axially and/or rotationally, to the user interface member, particularly to the user interface member body. The user interface member 1600 may be rigidly connected to the member 3010 via a connection interface 3020 (see FIG. 4D), e.g. a snap fit, such that relative rotational and axial movement is prevented between member 3010 and user interface member 1600. Consequently, via the connection interface 3020 axial force and rotational torque can be transferred from the user interface member to the member 3010. The member 3010 may protrude distally from the setting surface 1610.

FIG. 4B shows the user interface member 1600 when assembled on the basis of a perspective view. FIG. 4C shows a top view onto the delivery surface 1620 of the user interface member 1600. FIG. 4D shows a sectional view along the line B-B in FIG. 4C.

The user interface member 1600 comprises a first portion 1603 and a second portion 1604. The first portion 1603 is the portion which provides at least one exterior surface of the member, e.g. the setting surface or the delivery surface or both surfaces. The second portion 1604 provides the interface to the drug delivery device (unit), represented schematically by member 3010. Thus, the mechanical connection to the mechanism may be performed via that portion to which the conductor carrier 3000 is secured. The respective portion 1603, 1604 may be rigid. That is to say, the respective portion may be provided to withstand a mechanical load, e.g. force or torque, and be configured to transfer the load, e.g. to the dose setting and drive mechanism. The load may be a load for the dose setting and/or dose delivery operation. The load may be a user generated load acting on the delivery surface or the setting surface. The first portion 1603 may have a continuous portion which extends circumferentially around a central main axis of the user interface member 1600—the continuous portion may form the setting surface—and a radially extending interconnecting portion being connected to the continuous portions on opposite sides of the central main axis—the interconnecting portion may form the delivery surface 1620. The second portion 1604 is radially and/or inwardly offset relative to the setting surface 1610. The second portion 1604 may be distally offset relative to the delivery surface 1620. An interspace may be formed between the inner wall facing the delivery surface and the second portion. The first portion 1603 may define an interior space of the user interface member 1600 and/or delimit the member relative to the exterior. The second portion may be arranged in the interior space, either only partially as depicted or completely. An outer radial surface of the second portion may face a radial surface of the first portion. The main axis 1613 of the user interface member, when the user interface member 1600 is connected to the member 3010, may be the axis around which the user interface member rotates relative to the housing 10 of the device (not shown). The first portion 1603 and the second portion 1604 may be oriented along the axis 1613.

The first portion 1603 and the second portion 1604 are connected to one another via a connection portion 1606 or connection feature. The connection portion 1606 is radially oriented, e.g. a web. The connection portion may have a radial main direction of extension. The first portion 1603 or at least a part thereof (see further above), the connection portion 1606 and/or the second portion 1604 may be integrated into a unitary structure, e.g. a plastic and/or molded structure. The connection portion is rigidly connected to the first portion and the second portion. The connection portion 1606 is deformable, preferably elastically deformable, such that the first portion 1603 may be moved relative to the second portion 1604, e.g. in a limited manner. The connection portion may be angularly or rotationally deformable and/or axially deformable. Axial deformability is particularly suitable for the delivery signaling unit, whereas angular or rotational flexibility is particularly suitable for a setting signaling unit. When the first portion 1603 is moved relative to the second portion 1604 and the associated switch 1650 or 1660 is triggered during this movement from a rest position to a signaling position, the signal for indicating the respective operation may be generated. In response to the signal, the electronic control unit 1100 expediently switches the electronic system into the state of higher power consumption as has been discussed already. In order to allow axial deformation in the distal direction, there may be an axial interspace 3030 between member 3010, particularly a proximally facing surface thereof, and the connection portion 1606 as is apparent from FIG. 4D. Alternatively or additionally, an axial interspace is present between the first portion and the second portion for the axial deformability, e.g. between a distally facing surface of the first potion and a proximally facing surface of the second portion. However, other configurations may also be suitable. The angular flexibility may be facilitated by an angular interspace 1607 between the connection portion 1606 and a region of the second portion 1604. Angular interspaces may be disposed on both sides of the connection portion 1606 or on just one side. This is apparent from FIGS. 4E and 4F which show sectional views along the designated lines in FIG. 4G. FIG. 4H shows the sectional view along the line C-C in FIG. 4D. The connection portion 1606 enables limited movement of the first portion 1603 relative to the second portion 1604.

The first portion 1603 is preferably axially guided relative to the second portion 1604, e.g. in order to avoid a tilting during the (limited) relative axial movement. For this purpose a guide feature 1609 of the first portion 1603, e.g. a pin-like feature and/or a feature protruding axially (distally) from the inner surface, preferably at the proximal end of the user interface member 1600, can be provided. The guide feature 1609 interacts with a corresponding guide feature 1608, preferably defining a guide opening, of the second portion 1604. The axial guiding interface established between the guide feature and the corresponding guide feature expediently allows relative axial and/or rotational movement between the first portion 1603 and the second portion 1604. The guide features 1608 and 1609 may be in radial frictional contact with each other to provide the guiding functionality.

The user interface member 1600 is designed such that, due to the flexibility provided by the connection portion 1606, the first portion 1603 moves relative to the second portion 1604 due to a force or torque applied by the user to the setting surface and/or the delivery surface before the user exerted load is transferred to further components or members of the dose setting and drive mechanism via the user interface member, particularly its second portion 1604. In case of a manipulation of the setting surface 1610, the movement of the first portion is a rotation, e.g. in or counter to the dose setting direction, where the dose setting direction is the direction required to increase the size of the set dose. Thus, play between the first and the second portions may be used to generate a use signal or activation prompt signal such as a setting signal or a delivery signal depending on whether the setting switch 1660 or the delivery switch 1650 is triggered. If only a setting or delivery switch is present, of course only setting or delivery signals may be generated. The respective signal may be used to trigger the electronic control unit to switch the electronic system into the state of higher power consumption. Once the relative movement of the first portion relative to the second portion from a rest position has been completed, the first and the second portion may move in unison away from the rest position of the first portion, e.g. in the dose setting direction to set a non-zero dose or in the distal direction to deliver a dose. Alternatively, an activation operation different from the dose operation may be used, e.g. counter to the dose setting operation, in order to generate the signal as has been mentioned above.

As seen along a force transfer path from the exterior surface which is manipulated by the user for exerting the force to the member 3010, which expediently belongs to the dose setting and drive mechanism, the first portion 1603 is closer to the user than the second portion 1604. The connection portion 1606 is arranged between the two portions as seen along the force transfer path. In the depicted embodiment, the first portion provides the surface for interfacing with the user, whereas the second portion provides the interface to the dose setting and drive mechanism of drug delivery device unit or the drug delivery device.

The conductor carrier 3000 is axially and/or rotationally fixed to the second portion 1604. Consequently, the switches 1660 and 1650 are fixed to the second portion 1604. Features which are arranged to trigger the respective switch may be provided on the first portion 1603. In the depicted embodiment, an interface feature, e.g. a distally directed protrusion 1655, is arranged to engage a trigger feature 1657 of the delivery switch 1650. When the delivery surface 1620 is pressed in order to initiate a delivery operation, the (axially or distally directed) force is transferred to the trigger feature 1657 via the interface feature 1655 due to the axial flexibility which the connection portion 1606 provides and, via the trigger feature, to a load sensitive component of the switch 1650 which causes the switch to generate the signal (in this case a delivery signal). The delivery signal is expediently generated before the clutch interface is switched, e.g. disengaged. For example the signal may be generated before the member 3010 disengages a dial sleeve or number sleeve for a dose delivery operation, where the member 3010 and the dial sleeve or number sleeve are rotationally locked during the preceding dose setting operation for setting the dose to be delivered. The setting switch 1660 comprises an interface feature 1665, e.g. a recess, such as a radially oriented recess, provided in an inner surface of the first portion 1603. The interface feature 1665 is arranged to engage a trigger feature 1667 of switch 1660. When the user interface member 1600 is rotated via the setting surface 1610, the initial relative movement between the first portion 1603 and the second portion 1604 may cause that load is transferred to a load sensitive component of the switch 1660 via the trigger feature 1667 which causes the switch 1660 to generate the signal (in this case a setting signal).

The relative movement between the first portion 1603 and the second portion 1604 required for triggering the switch may be smaller than the rotational movement required to set a dose of one unit increment and/or than the axial movement required to switch the state of the clutch, e.g. to disengage two clutch components or to engage two clutch components, in the dose setting and drive mechanism, which switches that mechanism from the dose setting configuration into the dose delivery configuration. Once the user exerted load is removed from the respective surface of the first portion, the elasticity of the connection portion may re-establish the initial state, i.e. the initial or first relative position between the first portion and the second portion. The FIGS. 4D through 4G depict the arrangement in the initial state. When a dose is set starting from the situation in FIG. 4F by rotating user interface member relative to the housing, initially the first portion rotates relative to the second portion, e.g. clockwise and/or in the dose setting direction. During this phase, the signal may be generated by switch 1660. Also, the interspace 1607 may be closed or at least reduced, e.g. until the connection portion 1606 abuts the second portion 1604. After the signal has been generated, both portions 1603 and 1604 may co-rotate away from the signaling position until the desired dose has been set. For the delivery operation, the relative axial movement between the first portion and the second portion may trigger signal generation via switch 1650.

In the depicted embodiment, the axial force during the delivery operation is reacted by the carrier 3000. Consequently, it may be required that the entire dispensing force acts on the carrier. This can be avoided or its impact on the carrier 3000 can be at least reduced if an axial abutment between the first portion and the second portion is established once the first portion has been displaced relative to the second portion in order to trigger the switch 1650. The axial abutment may, for example, be achieved by one or more protrusions protruding radially from the guide feature 1609 outside of the guide opening (not explicitly shown) where a distally facing surface of the respective protrusion is arranged to abut a proximally facing surface of the second portion 1604. In the same manner, a rotational abutment may be provided between the first portion and the second portion, by two surfaces facing each other in the angular direction, after the relative movement between the two portions for generating the signal has been completed. This prevents excessive loads acting on the connecting portion which, on account of its flexibility, may be particularly prone to damages. To put it differently, after the relative movement between the first portion and the second portion (axial and/or rotational movement) has been completed, for further movement away from the rest position of the first portion relative to the second portion, a follower interface may be formed between the first portion and the second portion, wherein the follower interface slaves the second portion to the first portion in the direction which is defined by the movement of the first portion towards the second portion, e.g. the dose setting direction or the distal direction for dose delivery.

The relative rotational or angular movement between the first portion and the second portion required to generate the signal (e.g. the setting signal) may be less than or equal to the angle corresponding to one unit setting increment (e.g. 15°). For example the relative rotational or angular movement between the first portion and the second portion required to generate the signal is less than or equal to one of the following values: 15°, 10°, 9°, 8°, 7°, 6°, 5°. Alternatively or additionally, the relative rotational or angular movement between the first portion and the second portion required to generate the signal is greater than or equal to one of the following values: 1°, 2°, 3°, 4°. The relative axial movement between the first portion and the second portion required to generate the signal (e.g. the delivery signal) may be less than or equal to one of the following values: 1 mm, 0.9 mm, 0.8 mm, 0.7 mm, 0.6 mm, 0.5 mm. Alternatively or additionally, the movement may be greater than or equal to one of the following values: 0.2 mm, 0.3 mm, 0.4 mm. Consequently, the angular or axial distance between an abutment feature on the first portion and the corresponding abutment feature in the second portion may assume according values, in particular in the initial state or arrangement between the first portion and the second portion before the relative movement is commenced. The relative axial movement between the first and second portion required to trigger a signal may be 0.5 mm for example (i.e. the axial separation of the first and second positions). The relative angular movement between the first and second portion required to trigger a signal may be 4° for example (i.e. the angular separation of the first position or rest position and the second position or signaling position). Having a relative rotational or angular movement between the first portion and the second portion required to generate the signal of less than one half of the angle corresponding to the unit setting increment (the increment angle may be 15°) has the advantage that the signal generation can be incorporated into the dose operation easily. Having a relative rotational or angular movement between the first portion and the second portion required to generate the signal of more than one half of the angle corresponding to the unit setting increment has the advantage that the user may have the impression that he is doing something significant. Hence, the rotation to generate the signal may be 10°, for example. This may be particularly suitable for an activation operation different from the dose setting operation, for example, e.g. counter to the dose setting direction.

Having the angle to generate the signal less than the angle of the unit setting increment may be advantageous, as the rotational movement which triggers the signal may smaller for the user. However, we note that rotations which are greater than the angle corresponding to one unit setting increment, e.g. 20°, for generating the signal are also conceivable. The user may verify the current dose which is set via a dose display, e.g. via the number sleeve. Hence relative rotations between the first portion and the second portion greater than the angle corresponding to one unit setting increment to generate the signal are not problematic in this respect.

By integrating the connection portion 1606 into the user interface member, the user may operate the user interface member without any hint to the user that a relative movement between portions of the user interface member is used to control or switch the state of the electronic system such as to switch components or units of the system to a state of higher power consumption.

In one embodiment, the first portion is movable relative to the second portion in two opposite directions, e.g. axial and/or rotational directions. The signaling unit may be configured to provide the electrical signal only during the movement in one of these directions or in both opposite directions. In case a signal is provided in two opposite directions, one of these directions, e.g. the dose setting direction, could be used for providing the activation prompt signal or use signal to switch the system to the state of higher power consumption. The other direction may be used to provide a signal for a different purpose, e.g. to initiate a synchronization process for dose data with another electronic device, such as a computing device, preferably a mobile phone or server. The synchronization may be performed by a wireless interface established between the electronic system and the electronic device via the communication unit. This may be an option for the user to manually trigger a synchronization process by an activation operation. The synchronization process—or an attempt to conduct such a process—may be integrated by default into the operation routine of the electronic system after a dose delivery operation has been performed and the motion sensing unit has generated data.

In case the rotation counter to the dose setting direction is employed for signal generation, e.g. to manually trigger a synchronization event, the first portion may be rotated relative to a zero dose stop or zero unit stop which may engage the second portion and prevent rotation of the second portion in the same direction as the first portion, thereby enabling the rotation of the first portion away from the second portion.

Using a rotational movement for generating one or more signals indicative for a manipulation of the user interface member, wherein, in response to the respective signal, the electronic system performs operations or changes its state, may be advantageous over employing (only) axial movement for this purpose. This is because it is less likely that the user interface member is rotated accidentally than that it is displaced axially accidentally.

We note that, if the setting signaling unit and the delivery signaling unit are provided, the setting signaling unit may be used to activate the electronic control unit, e.g. to switch it from a dormant state to state of higher power consumption, in which the motion sensing unit and/or the communication unit is not yet active. Further units may be activated by the delivery signaling unit. Of course, is also possible to provide just the setting signaling unit or the delivery signaling unit. If the system is switched to the second state of higher power consumption by the setting signaling unit, the time between the delivery operation, i.e. when the motion sensing unit has to record data or generate data on the relative movement between elements of the dose setting and drive mechanism, is greater than when the delivery signaling unit is employed for this purpose. However, the delivery signaling unit is usually actuated closer to the delivery operation than the setting signaling unit. Hence, if the delivery signaling unit is used to switch the electronic system to the second state, an unnecessary switching to the second state is less likely to occur, as some users might play with setting and canceling doses rotating the user interface member in the opposite direction without actually conducting a delivery operation or manipulating the delivery surface. Utilizing the relative movement during setting or delivery seamlessly integrates the signal generation into the dose setting or dose delivery operation. The electronic system 1000 may be configured such that signals from the delivery signaling unit are only considered e.g. by the control unit, if a setting event has been determined before that signal via the setting signaling unit, preferably within a predetermined time interval, e.g. within less than one of the following values: 5 min, 3 min, 2 min, 1 min, 45 s, 30 s, 20 s, 15 s. Also, the delivery switch 1650 may be switched to a state of higher power consumption after the setting event has been detected by the switch 1660. Moreover, in case the user exerts simultaneous load on both surfaces, the setting surface and the delivery surface, which entails generation of setting and delivery signals the electronic control unit can issue an according signal, record an according event or generate an alarm that indicates that the system might have been handled improperly as both surfaces have been provided with load simultaneously.

It should be noted that, instead of having an integral portion of the user interface member which connects the first and second portions, all portions being integrally formed with one another in a unitary structure, the signal generation could also be realized with having two separate parts of the user interface member which are movable relative to one another, e.g. before setting the first unit setting increment has been completed, before delivering a dose, and/or before switching the clutch interface. The parts may be spring biased to permit the relative movements required for the signal generation. The unitary structure, however, is preferred as this renders it less likely that the user notices the multipart construction when operating the user interface member.

FIG. 5 illustrates another embodiment of an electronic system for a drug delivery device. In this embodiment, as in the previously described embodiment, a setting signaling unit (represented by switch 1660) is provided, which generates a signal upon manipulation (rotation) of the setting surface, which involves relative rotation between the first portion and the second portion. The signal may, preferably directly, trigger the electronic control unit 1100 to switch the electronic system to the second state. As the embodiment is very similar to the one previously discussed, the following description focusses on the differences.

Rotation of the first portion 1603 relative to the second portion and the switch 1660 (which, again, may be a rotationally operated force or torque switch) in one rotational direction triggers a signal generation. We note that when the first portion 1603 is rotated in a first rotational direction, here the clockwise direction, the trigger feature 1667, preferably immediately, transfers the load (force or torque) to the switch 1660 on account of the angular abutment between the first portion 1603 and the trigger feature 1667. Rotation of the first portion in the second rotational direction opposite to the first rotational direction relative to the first portion does not result in generation of a signal since the load is not transferred to the trigger feature 1667, e.g. on account of the interface feature 1665 allowing a relative rotation before an angular abutment is established with the trigger feature 1667. Hence, in the second rotational direction there is an angular clearance. Preferably, the second portion is slaved to the first portion rotationally in the second direction before the signal could be generated. We note that aside from a rotational clearance, the signal generation in the direction opposite to the first direction could also be prevented by various other measures. The first rotational direction which generates the signal may be the dose setting direction, i.e. the direction in which the user interface member is rotated when the set dose is increased. Alternatively, the first rotational direction may be the opposite rotational direction. In this case, an activation operation is employed for the signal generation which is different from the dose setting operation and the dose delivery operation, which may be advantageous as well.

Thus, in this embodiment, the user interface member 1600 may selectively generate signals, i.e. only when the user interface member is rotated in the first rotational direction. Such directional selectivity may also be applied in the previously discussed embodiment.

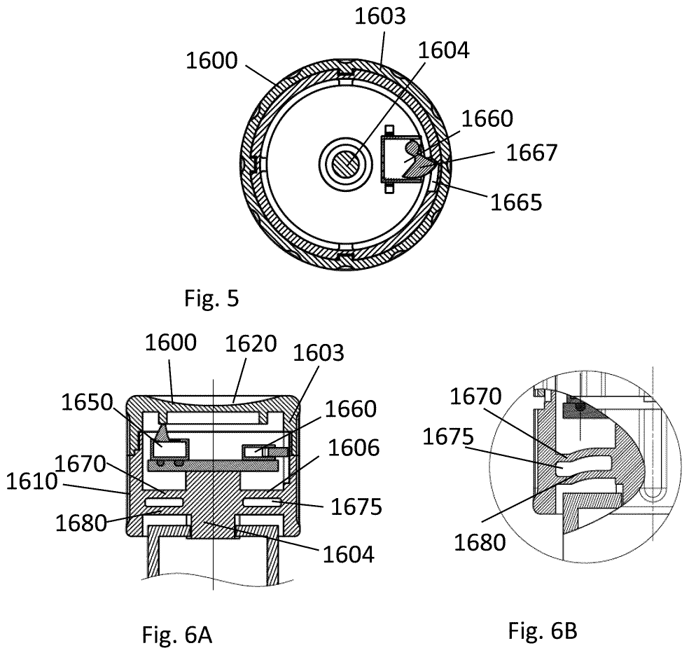

FIGS. 6A and 6B illustrate another embodiment of an electronic system. In general this embodiment is very similar to the one which has been discussed in conjunction with FIGS. 4A to 4H. Therefore, the following discussion focuses on the differences. This embodiment also employs two portions 1603 and 1604 of the user interface member 1600, which are connected to one another via the connection portion 1606. As opposed to the previously described embodiment, the connection portion 1606 has a plurality of regions 1670 and 1680. Each region is extending radially and/or connects the first portion 1603 to the second portion 1604. The regions may be webs. The regions are axially offset from each other. The regions 1670 and 1680 are axially separated from one another by an interspace or void 1675. In the detail shown in FIG. 6B, the (axial) flexibility of the connecting portion 1606 is illustrated, which involves deformation of both regions. The interspace or void 1675 may have a cross-section—e.g. taken along the main axis of the user interface member—which is shaped according to a rectangle or a non-rectangular parallelogram. This may assist in avoiding non-desired tilting movements of the user interface member 1600 in the radial and axial direction relative to the axis and/or relative to member 3010. Accordingly, this embodiment does not require a guiding functionality as the one described further above. Consequently, the guide features 1608 and 1609 are not provided in the depicted representation.

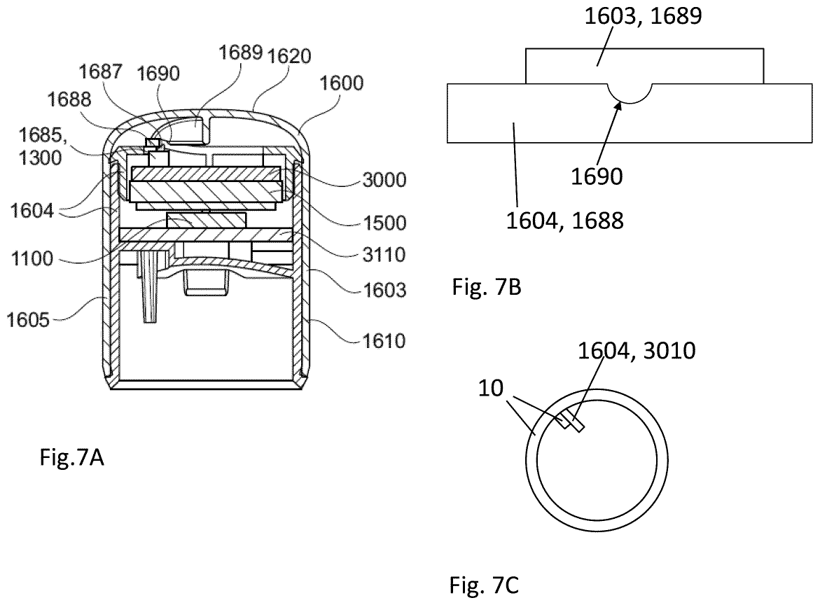

FIGS. 7A through 7C illustrate another embodiment of the electronic system. In general, this embodiment is similar to the ones previously described such that features which are disclosed in this context may also apply to the present embodiment, especially if these features are not contradictory to what is discussed in the following. In previously described embodiments, the signal generation by the signaling unit was sometimes associated with a manipulation or action which is directly associated with and/or integrated into the dose setting operation and/or the dose delivery operation, e.g. into unidirectional movements of the user interface member required for these operations. In the present embodiment, a distinct user action, i.e. an activation operation different from the dose setting operation and the dose delivery operation, is required for generating the signal, in particular for switching the electronic system into the state of higher power consumption, e.g. when the motion sensing unit and/or the communication unit is operational.

The user interface member 1600 is depicted in FIG. 7A on the basis of schematic sectional view. The user interface member 1600, again, has the first portion 1603 and the second portion 1604. In the interior of the user interface member 1600 the conductor carrier 3000 is provided. An electrical switch 1685, e.g. a micro switch such as a micro force switch, for the signaling unit 1300 is arranged on the carrier 3000. The switch 1685 has a trigger feature 1687, which, when subjected to a force, triggers the switch to generate the signal. The carrier, again, may be a printed circuit board or another conductor carrier. The switch 1685 is arranged below the delivery surface 1620. The signaling unit 1300 is configured to generate the signal when the first portion 1603 rotates relative to the second portion 1604. The switch 1685 is operatively coupled to the electronic control unit 1100 which is also housed within the user interface member body 1605. In the depicted situation, the control unit 1100 and the signaling unit, especially the switch 1685, are electrically connected to and/or mounted on different conductor carriers, i.e. the unit 1100 is connected to another conductor carrier 3005 than the switch 1685. The conductor carrier 3005 is also mounted in the interior of the user interface member body 1605. The conductor carriers 3000 and 3005 are expediently electrically interconnected, e.g. by a conductor, such as a metal pressing component. The conductor carriers may be arranged in a stacked and/or parallel manner. In this way, the signal triggered by the switch 1685 can be detected or processed by the control unit 1100. The power supply 1500, e.g. a coin cell battery, is arranged on that side of the conductor carrier facing away from the delivery surface 1620 and/or between the two conductor carriers 3000 and 3005. The respective carrier is expediently secured to the second portion 1604 of the user interface member 1600. The second portion 1604 may have a plurality of parts fixedly secured to each other (as illustrated) or just one part. The same holds for the first portion (not illustrated). In case the second portion is formed of different parts, one carrier may be fixed to one of the parts and the other carrier may be fixed to the other one of the parts. It should be noted, however, that the electronic control unit 1100 and the signaling unit 1300 or switch 1685 could also be arranged on the same carrier. When the switch 1685 is triggered, the motion sensing unit (not explicitly shown, e.g. on the same carrier as the control unit and/or the carrier closer to the distal end of the user interface member, which is the lower end in FIG. 7A) or another electrically powered unit may be woken and/or switched to a state of higher power consumption to switch the system to the second state of higher power consumption as has been discussed further above already.

The first portion 1603 and the second portion 1604 are, in the depicted embodiment, formed by separately fabricated parts which are assembled to one another. In the depicted embodiment, the first portion and the second portion are coupled to one another via a force-sensitive coupling 1690. The force-sensitive coupling 1690, preferably, locks the first portion and the second portion together such that the first portion and the second portion move, e.g. rotate, together unless a force acts on the coupling which is greater than a predetermined threshold force, e.g. a force up to which the coupling is stable. In the present case, the threshold force may be greater than regular forces occurring during dose setting and dose delivery. That is to say, the force-sensitive coupling may be stable and transfer force or torque during regular movements of the user interface member, e.g. for dose setting and/or for dose delivery, relative to the housing 10. Particularly, if a force or torque below the threshold force or torque is exerted, the first portion and the second portion act as a single part. However, in case a force or torque greater than the threshold force or torque is exerted onto the coupling, the coupling is released and the first portion may move relative to the second portion. The force-sensitive coupling is expediently configured such that the first portion 1603 and the second portion 1604 co-rotate unless the force or torque acting on the coupling exceeds the threshold force or torque. When this threshold force or torque is exceeded the coupling may be released and there may be relative rotation between the first portion 1603 and the second portion 1604. Particularly, the first portion 1603 which exhibits the setting surface 1610 may then be rotated relative to the second portion 1604. This relative rotation can be used to trigger the signaling unit 1300 to provide the signal to switch the electronic system into the state of higher power consumption. The force-sensitive coupling 1690 may be formed by a connection feature of the first portion and a connection feature of the second portion interacting, e.g. engaging, to establish the force-sensitive coupling. The force-sensitive coupling 1690 is, very schematically, depicted in FIG. 7B by a protrusion on the first portion 1603 engaging an indentation on the second portion 1604. However, it should be appreciated that further configurations are possible and that this implementation is merely an example.

The force-sensitive coupling is expediently dimensioned such that forces or torques occurring during regular setting events are transferred via the coupling from the user interface member 1600 to the dose setting and drive mechanism, e.g. to member 3010 described previously (see FIG. 4A, for example) to which the user interface member may be connected. The forces or torques transferred via the force-sensitive coupling may be the forces or torques required to rotate the user interface member 1600 in a dose setting direction, which, in the depicted setting, would be clockwise as seen in top view onto the delivery surface 1620, until a dose is set. Also, forces or torques for canceling or reducing a previously set does which involve rotation in a direction opposite to the dose setting direction are expediently transferred via the force-sensitive coupling. That is to say the first portion 1603 and the second portion 1604 are rotationally locked due to the force-sensitive coupling for dose setting and for canceling or reducing a previously set dose. For the activation operation, the force-sensitive coupling is released.

The force-sensitive coupling may transfer forces or associated torques which are less than or equal to 0.05 Nm or less than or equal to 0.04 Nm. The torque may be greater than 0.005 Nm. These forces or torques are typical for a dose setting operation, e.g. to set a dose, to adjust a set dose or to cancel a set dose. Typical dialing or setting forces or torques range from 0.014 Nm to 0.03 Nm, e.g. for the device (unit) discussed in conjunction with FIG. 1. Thus, if forces or torques within the specified region or ranges are applied to the setting surface, these forces or torques may be transferred via the force-sensitive coupling without involving relative rotational movement of the first and second portions. If greater forces or torques are applied, this will result in the relative movement between the first portion and the second portion.

The force-sensitive coupling 1690 may be released in order to enable relative rotation between the first portion 1603 and the second portion 1604. The coupling may be released only when a force or associated torque greater than or equal to one of the following values acts on the force-sensitive coupling: 0.05 Nm, 0.06 Nm, 0.07 Nm, 0.08 Nm, 0.09 Nm, 0.1 Nm.

In an initial position of the user interface member, where no dose has been set by the user (zero dose position), rotation of the user interface member 1600 in the direction opposite to the dose setting direction with respect to the housing 10 and/or another component or member of a drug delivery device may be prevented by way of a stop engagement. This engagement is often also termed zero unit stop or zero dose stop. In the present embodiment, the stop engagement can be implemented or is implemented via the second portion 1604 of the user interface member 1600 or a member connected to the portion, e.g. member 3010 of the dose setting and drive mechanism. The stop engagement is very schematically illustrated in FIG. 7C. Here, a stop feature of the portion 1604 or the member 3010, abuts a corresponding stop feature which may be provided on the housing 10 or on another component of the device which is rotationally locked to the housing 10 during dose setting or at least at the beginning of dose setting against rotation in the direction opposite to the dose setting direction. In the present arrangement, the dose setting direction is the clockwise direction where the portion 1604/the member 3010 would be rotated away from the corresponding stop feature on the housing. The stop engagement may be established in the rest position of the first portion. When the user attempts to rotate the user interface member in the direction opposite to the dose setting direction, i.e. counterclockwise in the depicted configuration, the second portion 1604 will be prevented to follow that rotation on account of the stop engagement. Therefore, if the user exerts a force or torque via the setting surface 1610, the user exerted force or torque exceeding the threshold force or torque which can be transferred via the force-sensitive coupling 1690, the first portion 1603 will rotate relative to the second portion. This rotation can be used to trigger the switch 1685. The rotation of the first portion relative to the second portion, e.g. away from the rest position or zero dose position and/or counter to the dose setting direction, may be limited, e.g. by a rotational stop engagement, which may be formed by one feature on the first portion engaging one feature on the second portion rotationally (not explicitly shown). As the second portion cannot be rotated in the direction counter to the dose setting direction, the first portion, when the rotational stop engagement is established can also no longer be rotated in that direction.

The first portion 1603 of the user interface member 1600 comprises a trigger feature 1689. This feature is implemented as a protrusion in the depicted embodiment, e.g. an axially oriented protrusion, such as a protrusion with a free distal end. This trigger feature may move relative to the switch 1685 when the first portion moves relative to the second portion and directly (not shown) or indirectly via a further feature interact with the switch to trigger the switch 1685. The first portion may be coupled to the second portion such that relative rotational movement of the first portion relative to the second portion is converted into axial movement of a further feature for triggering the switch.

In the depicted embodiment a flexible arm 1688 is provided, e.g. a trigger arm to trigger the switch 1685. The arm may be the further feature which is displaced axially to trigger the switch. The arm is fixedly connected to or integrated into the second portion 1604 in the depicted embodiment. The arm may be oriented angularly. The arm 1688 is expediently displaceable or deformable, e.g. axially, to trigger the switch, e.g. via contact with the trigger feature 1687. The arm 1688 has a free end delimiting the arm in the direction of rotation of the first portion 1603 relative to the second portion 1604 from the rest position to the signaling position, e.g. counter to the dose setting direction. The arm 1688 preferably is designed to interact with the trigger feature 1687 of the switch 1685. If the first portion 1603 rotates towards the free end of the arm, which is counterclockwise in the depicted configuration, the arm 1688 will deflect axially, e.g. distally, thereby causing triggering of the switch 1685.

The arm 1688, preferably, is elastically displaceable such that the elastic restoring force may be used to reestablish its original position and/or reestablish the force-sensitive coupling when the user releases the user interface member. That is to say, the bias may move the first portion into its rest position relative to the second portion such that the initial state of the user interface member is reestablished, e.g. when the user releases the setting surface 1610. Alternatively, another biasing mechanism for reestablishing the force-sensitive coupling or the initial state may be provided, e.g. a separate spring mechanism. Also, the triggering of the switch may be achieved by a different mechanism such as directly via the trigger feature of the first portion interacting with the switch such as the trigger feature 1687 thereof. It will be appreciated that a construction with the arm 1688 being fixedly connected to or integrated into the first portion 1603 could also be implemented.

Once the switch 1685 has been triggered, an according signal may be detected by the electronic control unit 1100, which, in turn, may issue a command to switch the motion sensing unit into the state of higher power consumption, e.g. as a dose setting and/or dispensing operation is to be expected soon. That is to say, in this case, an attempt to rotate the user interface member in a direction counter to the dose setting direction when the user interface member is in the initial position may be used to generate the activation prompt or use signal. If, within the predetermined time interval, e.g. within 30 seconds, after the motion sensing unit has been switched to the state of higher power consumption, no movement is detected by the motion sensing unit, the electronic system can be switched, again, to the state it had before, e.g. it may be switched off again, in order to avoid unnecessary power drain.

In one embodiment, the electronic system is switched only to the second state of higher power consumption, when the first portion is kept away from the rest position, e.g. in the signaling position, for more than a predetermined time, e.g. 3 s or more or more than 5 s. Hence, the electronic control unit, or another unit of the system, may evaluate a time characteristics of the signal(s) generated by the signaling unit, e.g. the signal duration or the time between two successive signals. If the time characteristic meets a criterion indicating that the first portion was away from the rest position for more than the predetermined time, the system can be switched to the second state. Using the predetermined time is particularly suitable for characterizing an activation operation. The electronic control unit may issue the signal or command to switch the system to the second state, e.g. to activate the communication unit and/or the motion sensing unit only if the evaluation of the time characteristic indicates that the first portion has been away from the rest position and/or in the signaling position for a time exceeding the predetermined time. For example, the predetermined time can be used to distinguish the activation operation form the dose operation. If the predetermined time is used, it is facilitated that the force required for the activation operation can be similar to or even smaller than the force required for the dose operation, e.g. the force for the dose setting operation. In the present embodiment, the force the user has to exert to release the force-sensitive coupling with a rotation counter to the dose setting direction, may be the same or smaller than the force required to set a dose by moving the first portion and the second portion in the dose setting direction (in FIGS. 7A to 7 the setting direction is the direction to move the first portion and the second portion away from the signaling position). The activation operation can be used to manually trigger a synchronization operation by way of the communication unit.

It should be noted that the described time-based approach is not restricted to this embodiment, but can be applied in the other embodiments as well, especially if an activation operation different from the dose operation is used to trigger switching of the system to a state of higher power consumption. For example, the activation operation may be an operation, where the delivery surface 1620, e.g. in the setting shown in FIGS. 4A to 4H, is pressed and held for the predetermined time, e.g. when a zero dose is set or with a force smaller than is required for initiating the dose delivery operation. The same holds for a rotation counter to the dose setting direction for an activation operation, where the first portion needs to be held away from the rest position for a predetermined time before the system is switched to the second state. Alternatively or additionally, if, in FIGS. 4A to 4H, the rotation counter to the dose setting direction is used to generate the signal, e.g. for an activation operation and/or in the zero dose set position, the force to do so may be similar to the force for dose setting. The rotation angle required for signal generation can be greater than the angle corresponding to one unit setting increment and/or greater than or equal to 10°, 15°, 17°, 20°. Having a greater rotation angle renders it less likely that the signal is generate accidentally.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N-(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrom.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present disclosure include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present disclosure, which encompass such modifications and any and all equivalents thereof.

An example drug delivery device may involve a needle-based injection system as described in Table 1 of section 5.2 of ISO 11608-1:2014(E). As described in ISO 11608-1:2014(E), needle-based injection systems may be broadly distinguished into multi-dose container systems and single-dose (with partial or full evacuation) container systems. The container may be a replaceable container or an integrated non-replaceable container.

As further described in ISO 11608-1:2014(E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1:2014(E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1:2014(E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

The scope of protection is not limited to the examples given herein above. Any disclosure disclosed herein is embodied in each novel characteristic and each combination of characteristics, which particularly includes every combination of any features which are stated in the claims, even if this feature or this combination of features is not explicitly stated in the claims or in the examples.

### REFERENCE NUMERALS

1 injection device, drug delivery device or device unit
10 housing
12 dosage knob
11 injection button
13 window
14 container
15 needle
16 inner needle cap
17 outer needle cap
18 cap
70 dial or number sleeve
71a-c formation
1000 electronic system
1100 electronic control unit
1200 motion sensing unit
1300 signaling unit
1310 switch
1400 communication unit
1500 electrical power supply
1600 user interface member
1601 part
1602 part
1603 portion
1604 portion
1605 user interface member body
1606 connection portion
1607 interspace
1608 guide feature
1609 guide feature
1610 setting surface
1613 axis
1615 connection feature
1620 delivery surface
1650 delivery switch
1655 interface feature
1657 trigger feature
1660 setting switch
1665 interface feature
1667 trigger feature
1670 region
1675 interspace
1680 region
1685 switch
1687 trigger feature
1688 arm
1689 trigger feature
1690 force-sensitive coupling
3000 conductor carrier
3005 conductor carrier
3010 member
3020 interface
3030 interspace

1. An electronic system for a drug delivery device, the electronic system comprising:
a user interface member configured to be manipulated by a user for performing a dose operation, the user interface member comprising a first portion and a second portion, the first portion being movable relative to the second portion from a rest position to a signaling position;
an electronic control unit configured to control operation of the electronic system, the electronic system having a first state and a second state, wherein the electronic system has an increased electrical power consumption in the second state as compared to the first state;
an electrical signaling unit configured to provide an electrical signal indicative that the user interface member is being manipulated;
wherein the electronic control unit is configured to switch the electronic system from the first state into the second state in response to the electrical signal; and,
wherein the electrical signaling unit is configured to provide the electrical signal in response to a movement of the first portion relative to the second portion from the rest position into the signaling position.

2. The electronic system of claim 1, wherein the movement of the first portion from the rest position to the signaling position involves rotational movement of the first portion relative to the second portion.

3. The electronic system of claim 1, wherein the electronic control unit is connected to the second portion.

4. The electronic system of claim 1, wherein the first portion and the second portion are permanently directly mechanically interconnected during the dose operation by a mechanical interconnection.

5. The electronic system of claim 4, wherein the mechanical interconnection comprises a connection feature that is integrally formed with the first portion and the second portion.

6. The electronic system of claim 5, wherein the connection feature is flexible for permitting movement of the first portion relative to the second portion from the rest position to the signaling position.

7. The electronic system of claim 4, wherein the mechanical interconnection comprises a connection feature of the first portion and a connection feature of the second portion that interact with each other to establish the mechanical interconnection.

8. The electronic system of claim 7, wherein the mechanical interconnection is releasable for conducting an activation operation that involves the movement of the first portion from the rest position into the signaling position, the activation operation being different from the dose operation.

9. The electronic system of any one of claim 7, wherein the mechanical interconnection is configured such that a force or torque that a user has to exert on the first portion to perform the dose operation with the user interface member is less than a force or torque that the user has to exert on the first portion to move the first portion from the rest position into the signaling position.

10. The electronic system of claim 1, wherein the second portion is configured to abut a stop feature when the first portion is moved from the rest position towards the signaling position.

11. The electronic system of claim 1, wherein the rest position is a no dose set position.

12. The electronic system of claim 1, wherein the signaling unit comprises an electrical switch mechanism that is configured to be triggered to provide the electrical signal when the first portion moves relative to the second portion from the rest position to the signaling position.

13. The electronic system of claim 1, wherein the electronic system is configured such that the electronic system is switched only from the first state into the second state when the first portion of the user interface member is held away from the rest position for a time exceeding a predetermined time.

14. The electronic system of claim 1, wherein, when the first portion is in the signaling position, the first portion is biased towards the rest position by a biasing mechanism of the user interface member.

15. The electronic system of claim 1, wherein the electronic system is configured as an add-on module for a drug delivery device unit.

16. (canceled)

17. The electronic system of claim 1, wherein the first portion and the second portion are coupled via a force-sensitive coupling.

18. The electronic system of claim 1, wherein the system is configured such that the first portion is connected to the second portion in a torque-proof and/or force-proof way after the relative movement has been completed in the direction away from the rest position.

19. The electronic system of claim 1, wherein the electronic system comprises one or more of
an electrical motion sensing unit;
a communication unit;
a memory unit.

20. The electronic system of claim 1, wherein when in the rest position, the first portion is movable relative to the second portion in two different directions.

21. A drug delivery device comprising:
an electronic system having a first state and a second state, wherein the electronic system has an increased electrical power consumption in the second state as compared to the first state;
the electronic system comprising:
a user interface member configured to perform a dose setting operation to set a dose of drug to be delivered by the drug delivery device and/or a dose delivery operation for delivering a set dose, the user interface member comprises a first portion and a second portion, wherein the first portion is movable relative to the second portion from a rest position of the first portion to a signaling position of the first portion;
an electrical signaling unit configured to provide an electrical signal indicative that the user interface member is being manipulated; and
an electronic control unit configured to control operation of the electronic system and to switch the electronic system from the first state into the second state in response to the electrical signal,
wherein the electrical signaling unit is configured to provide the electrical signal in response to the movement of the first portion relative to the second portion from the rest position into the signaling position; and
a reservoir with a drug.
